(19) United States
(12) Patent Application Publication
(10) Pub. No.: US 2023/0082966 A1
(43) Pub. Date: Mar. 16, 2023

(54) DEVICES AND METHODS FOR PREDICTING, IDENTIFYING AND/OR MANAGING PNEUMONIA OR OTHER HEALTH STATUS

(71) Applicant: Respirix, Inc., San Francisco, CA (US)

(72) Inventors: Daniel R. BURNETT, San Francisco, CA (US); Alexander VERGARA, San Francisco, CA (US); Michael JAASMA, San Francisco, CA (US); Cory SYLLIAASEN, San Francisco, CA (US)

(73) Assignee: Respirix, Inc., San Francisco, CA (US)

(21) Appl. No.: 17/933,403

(22) Filed: Sep. 19, 2022

Related U.S. Application Data
(63) Continuation of application No. PCT/US2021/023185, filed on Mar. 19, 2021.
(60) Provisional application No. 62/994,629, filed on Mar. 25, 2020.
(60) Provisional application No. 63/137,593, filed on Jan. 14, 2021.

(51) Int. Cl. A61B 5/097; A61B 5/0205; A61B 5/1455; A61B 5/00; A61B 7/00; G16H 50/30

(52) U.S. Cl. CPC A61B 5/097; A61B 5/02055; A61B 5/14551; A61B 5/682; A61B 7/003; G16H 50/30; A63B 2230/42

(57) ABSTRACT

Devices and methods for predicting, identifying and/or managing pneumonia are disclosed and may generally comprise a housing, a mouthpiece in communication with the housing and configured for insertion within a mouth, a temperature sensor positioned within or along the housing, a pulse oximeter sensor positioned along the housing for sensing a heart rate or blood oxygen level, a microphone positioned within or along the housing for obtaining sounds associated with respiration, and a controller configured to receive physiologic parameters including the temperature, heart rate, blood oxygen level, and sounds associated with respiration. The controller can determine a likelihood of contracting pneumonia, a presence of pneumonia, or a status of pneumonia within the user based on deviations from a threshold value which are present in at least two of the physiologic parameters.

### CROSS-REFERENCE TO RELATED APPLICATIONS

This application is a continuation of International App. PCT/US2021/023185 filed Mar. 19, 2021, which claims the benefit of priority to U.S. Prov Applications 62/994,629 filed Mar. 25, 2020 and 63/137,593 filed Jan. 14, 2021, each of which is incorporated herein by reference in its entirety.

### FIELD OF THE INVENTION

The present invention relates to the field of predicting, detecting and monitoring patient health.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND OF THE INVENTION

Heart failure (HF) is the leading cause of hospitalization among adults over 65 years of age in the United States. In 2014, more than 5.1 million people in the United States were living with a diagnosis of HF, and as many as one in nine deaths each year can be attributed to complications stemming from this disease. Acute decompensation is a life-threatening consequence of HF that occurs when uncontrolled fluid retention in the thoracic cavity prevents the heart from maintaining adequate circulation. An important component of managing HF patients is maintaining an appropriate fluid volume by adjusting the patient's medications in response to his/her cardiac function. Fluid volume metrics, such as dyspnea, edema, and weight gain, can be monitored by patients at home as an indirect indicator of worsening cardiac function, but are highly non-specific and cannot predict decompensation risk with sufficient resolution to affect the hospitalization rate. Recent evidence has shown that directly monitoring cardiac function via an implantable sensor can provide clinicians with a remote monitoring tool to determine when medication adjustments can prevent decompensation and the need for hospitalization. However, the cost and invasive nature of these sensors severely restrict their potential for clinical adoption.

Various mechanisms have been employed to determine cardiac function and health. These include invasive technologies such as the Swan Ganz catheter and a pulmonary artery implant to less invasive technologies such as arterial waveform monitoring devices, and surface worn technologies such as bioimpedance monitors and noncontact technologies such as scales to monitor weight. The invasive technologies are more accurate but also more risky while the noninvasive technologies have less risk but are more cumbersome and typically less accurate. The presence of collected fluid, peripheral edema, ascites, pleural effusions and weight can also be used to monitor cardiac function in cardiac heart failure patients, but these parameters are merely symptomatic surrogates with poor correlation to actual cardiac output.

What is needed is a simple, repeatable, accurate monitor of cardiac function and other physiologic parameters that allows consistent measurement of cardiac output in the clinic, hospital and/or home environment. The present invention provides an easy to use, home-based device and method for the tracking of cardiac output, stroke volume and cardiac function. The invention can also be used for monitoring mechanical phases of the cardiac cycle, which are useful for diagnosing structural issues such as heart valve pathologies.

### SUMMARY OF THE INVENTION

The present invention is a non-invasive respiratory monitor that is capable of directly monitoring cardiac function in a remote setting. The respiratory monitor, or airway device/controller, detects minor variations in expiratory airflow pressure known as cardiogenic oscillations (COS), which are generated by changes in the pulmonary blood volume that correspond with the cardiac cycle. The strength, or magnitude, or variations in magnitude, of cardiac oscillations is a direct indicator of cardiac function and is directly correlated with stroke volume and inversely proportional to pulmonary artery pressure.

Minor, cyclic waveforms caused by cardiogenic oscillations, or cardiac pulses, can be detected in the bulk pressure and flow measurements of expiration and inspiration. The method and device of the present invention utilizes this ability to detect and isolate cardiac oscillations, or pulsations, within the sensed pressure profile in the airway of an animal or human. Pressure measured at around 100 Hz, or around 80 Hz to around 120 Hz, within the airway of a subject allows for excellent resolution of the pressure signal. When pressure in the airway is measured at or around this frequency, cardiogenic oscillations may be visible in the resulting pressure curve. These pulsations are best seen at end expiration, or during a breath hold, but can be seen throughout the breathing cycle. This result may be the result of the heart beating in close proximity to the lungs, which subsequently transmits the pressure fluctuations through the trachea to the mouth and nose. It may also be the result of pulmonary blood flow, which may slightly compress the lungs as the heart beats.

The magnitude of cardiac oscillations is indicated by the standard deviation, or variations, of the cardiac oscillation pressure waveform and is a direct indicator of cardiac function and is directly correlated with stroke volume and inversely proportional to pulmonary artery pressure. The cardiac performance of patients with heart failure is reduced when compared to that of healthy individuals, which will dampen the cardiac oscillation curve relative to healthy subjects.

In one embodiment of an airway device as described herein, the device may generally comprise a mouthpiece section and an opening section defining one or more airway lumens therethrough. The airway device may further comprise a first sensor in fluid communication with the one or more airway lumens and configured to detect an airway pressure when a user inhales or exhales through the one or more airway lumens, a second sensor positioned upon a hand-piece for contact against a portion of the user and configured to detect a physiological signal from the user, and a controller in communication with the first and second sensors, wherein the controller is programmed to correlate pressure oscillations in the airway pressure received from the first sensor with heartbeats received from the first sensor, the second sensor, or pressure data corresponding to a rough airway pressure.

In one embodiment of a method of correlating physiologic parameters, the method may generally comprise detecting via a first sensor an airway pressure of a user while inhaling or exhaling through one or more airway lumens of a respiration device having a mouthpiece section and an opening section, detecting via a second sensor positioned upon a hand-piece of the respiration device a physiological signal sensed from the user in contact with the second sensor, and correlating via a controller pressure oscillations in the airway pressure received from the first sensor with a timing of heartbeats received from the first sensor, the second sensor, or pressure data corresponding to a rough airway pressure.

The present invention senses pressure and/or flow within the airway by exposing the airway (via the patient's nose or mouth) to one or more pressure, flow, and/or other sensor(s). When the epiglottis is opened, this exposure to the airway allows pressure and/or flow sensors to detect small pulsations that occur during heart function. These fluctuations may also be detected with a sensitive enough sensor, when the epiglottis is closed. With an appropriately sensitive sensor sampling at a rapid frequency, waveforms can be seen in the airway corresponding to contractions, relaxation and valve openings in the heart. This phenomenon has been found to be repeatable and allows not only for tracking of heart and lung function and/or conditions (i.e. pulmonary edema, pleural effusions, congestive heart failure, aortic insufficiency, mitral, pulmonic, tricuspid insufficiency, etc.) but can be used to diagnose disease in patients using the airway device. Whereas electrocardiogram (ECG) is used to monitor and diagnose heart conditions based on the electrical signal being sent to the heart, the present invention provides additional information based on the actual mechanical function of the heart.

Preferably, the amplitude and/or area under the curves for pressure and/or flow data can be used to determine relative pulmonary blood flow, relative stroke volume, and/or relative pulmonary artery pressure. For example, as pulmonary blood flow increases, the amplitudes of the flow pulsations in the breath increase. Additional parameters, such as the slope of the pressure curve, changes in the curve or standard deviation of the curve can also be used to determine relative cardiac function. When tracked over time, these parameters provide noninvasive insights into the patient's changing cardiac health and can be used to adjust his/her care accordingly. This is particularly useful for people who are being monitored regularly for changes in their conditions, such as patients with heart failure. Patient pressure/flow curve data can also be compared to those of healthy or unhealthy patient populations to asses a particular patient's, or a group of patients', health.

In some embodiments, the patient is prompted by a controller to breathe into the device naturally for several cycles. This may be done automatically by a controller. Further, the airway device may be simply placed in the mouth and worn while going about activities of daily living to allow for natural sensing of respiratory rate and/or other parameters such as airway pressure, another powerful predictor and indicator of progressing illness. In some embodiments, the airway device/controller can calculate the rate of exhalation and capture cardiogenic oscillations at the same phase of breathing for each patient to allow for consistent measures of cardiac output and lung function. In other embodiments, the mean or median of the samples may be used as the representative value for that particular measurement. For example, the patient may breathe regularly for 2, 5, or 10 minutes, during which the pressure, flow, and other signals are captured, and at the end the of the session values such as the average amplitude of the signal caused by cardiogenic oscillations may be reported. In this way intra-measurement variability is reduced and the signal-to-noise ratio is improved.

Further, in some embodiments, the patient may be prompted by a controller to inhale deeply and hold his/her breath (or, if used in conjunction with a ventilator, the ventilator can be paused at end inhalation, end exhalation, or elsewhere, either manually, via the ventilator settings, or automatically with communication between airway device/controller and the ventilator or incorporation of airway device/controller into the ventilator) to see the impact of breathing on the pressure waveform. Variability in the respiratory pulse pressure waveform can be used to determine hydration status, as well as volume status. Dehydrated or hypovolemic patients will see a pulse pressure waveform that varies throughout the respiratory cycle due to the change in cardiac function with the changing thoracic pressures found with respiration. As fluid status is restored, this variability is reduced and lack of variability can provide a powerful indicator that fluid status has been restored. In addition to pulse pressure variability, heart rate variability may also be used to assess fluid status. Variability may be assessed on a continuous basis during natural or mechanical ventilation or may be assessed during a respirator pause to look for changes at end-inspiration and/or end-expiration over time to track variability. The ratio of end-inspiratory to end-expiratory pulse amplitude during respiration or with a breath hold may be determined. Variations in waveform peak-to-peak period and magnitude, in addition to other parameters, may be determined.

In some embodiments, the patient may be prompted by a controller to exhale against resistance, while leaving his/her throat open (i.e., leaving the glottis and/or epiglottis open). This is referred to as a “modified Valsalva maneuver” or MVM. The patient/user may be prompted to exhale within a specific pressure range and for a specific time period. For example, the user may be prompted to exhale at a pressure of 10 mm Hg (±0.5 mm Hg) for at least 5 seconds. “Exhaling” may include exhaling into a closed or open system. If the system is open, for example, with a resistance control orifice, the resistance control orifice must be small enough to allow the user to exhale at the given pressure for the given time frame. The user may be prompted by the controller, or instructed, to perform the MVM within the proper parameters (open throat, pressure, and time).

A respiratory pause may also be used to provide another determinant of cardiac output—change in end-tidal CO2 after a respirator pause. The use of respiratory pulse pressure waveform analysis in conjunction with the end tidal CO2 method may improve the accuracy of the results and make this method less susceptible to pulse pressure variability.

In addition, actual, or absolute, cardiac output can be determined without calibration using the airway device/controller. By combining the airway device/controller with spirometry or a ventilator, the volume of air in the lung can be accurately estimated. In addition, actual, or absolute, cardiac output can be determined using a CO2 sensor to determine end tidal CO2, as well as an air flow sensor and oxygen sensor. The calculations to determine cardiac output can be performed as described in “Noninvasive Monitoring Cardiac Output Using Partial CO2 Rebreathing” by Brian P. Young, MD, and Lewis L. Low, MD. A spirometer and/or ventilator may be stationary or ambulatory, or may be miniature and built into the mouthpiece itself.

In another embodiment, absolute stroke volume, cardiac output, and/or pulmonary artery pressure can be estimated by comparing the amplitude of the pressure or flow curves in the airway to the volume of air in the lungs and using correlation coefficients based on patient based variables such as their gender and height, in a similar manner to the way correlation coefficients can be use with pulse-transit-time to estimate blood pressure (see Gesche, Heiko, et al. “Continuous blood pressure measurement by using the pulse transit time: comparison to a cuff-based method.” (2011)). In this manner, the present invention may be used to estimate the actual volume displaced in the lung by the cardiac pulse, which represents the true stroke volume. An ECG or pulse oximetry signal may be used to help determine the pulse transit time.

Furthermore, in the setting of low pulse pressure variability this technique can also be used to calculate the dead space in the lung. This can be done by comparing the cardiac pulse pressure waveform at end-inhalation and end-exhalation. If tidal volume is known (i.e. with spirometry or mechanical ventilation), then, assuming the cardiac pulse is a constant, one can calculate the dead space in the lung by looking at the magnitude of the cardiac pressure pulse and calculating the predicted amplitude of the cardiac pulse, measuring the actual amplitude of the cardiac pulse, and determining the dead space information from the difference between the two (due to the extra dead space being compressed also). Total lung volume may also be calculated by the application of a fixed amount of analyte or a small bolus of gas/air to the lung then calculating the resulting concentration of the analyte or the final pressure after delivery of the bolus of air (assuming a breath hold at end-inspiration).

Due to its ease of use and non-invasive nature, the present invention lends itself well to home healthcare monitoring. In a preferred embodiment, the airway device will be handheld or body worn (but does not need to be). The airway device may continuously or intermittently measure flow rates/volumes, pressure, temperature, and/or gas concentrations in the airway. Patient manipulations may be requested by the airway device/controller (i.e. “Breathe deep then hold your breath for 5 seconds”) and the airway device/controller may be able to automatically or manually communicate the extracted information to the patient and/or healthcare provider, or with a mobile device, computer, server or other device. Alerts may be programmed into the airway device and/or controller, as well, to warn of impending issues or danger, or to guide the user through its use. By continuously sensing the pressure, the airway device/controller may also provide continuous feedback on the adequacy of the patient manipulations (i.e. “Slow down the speed of your breath”) to optimize the patient manipulations for improved data capture. Alerts may be audible, visual, vibration, etc. Alerts may also be sent to a physician, monitor, hospital, electronic health/medical records etc. Alerts may be transferred wirelessly to any device including a mobile device, computer, server, etc.

In temperature-sensing embodiments, the airway device/controller may sense inhaled and exhaled temperature and the controller, based on flow/heat exchange algorithms, reports the patient's temperature. Alternatively, the airway device/controller may report trends in temperature based on baseline data acquired when the patient was at a normal temperature. This deviation from baseline data can be utilized with any of the sensed parameters thereby allowing for the determination of a relative change in any of the parameters without knowing the actual value of any of the parameters.

In any of the home health, clinic or hospital embodiment of the airway device/controller of present invention, additional functionality may be incorporated, including temperature sensing, respiratory function monitoring (i.e. spirometry), acoustic monitoring (to track wheezing in asthmatics, etc.), detection of analytes and/or compounds in the breath (i.e. urea, markers of infection, O2, CO2, water vapor, etc.), detection of analytes in the saliva (since the device may be placed inside the mouth in some embodiments). Additional air sensors may include alcohol, and/or other drugs such as narcotics, marijuana, tobacco, etc.

In addition, physical sensors in contact with the body, for example the lips, fingers, hands, may include ECG sensors, pulse sensors, mucosal contact sensors, etc. When ECG sensors are in place, sampling of the pulsatile signals in the breath from the cardiogenic oscillations may be synchronized with the ECG signal in order to identify periodic signals, evaluate only the relevant portions of the signal and to reduce the amount of noise. For example, the magnitude of change in the pressure and/or flow signals during a set amount of time (such as 200 or 500 ms) may be the variable of interest that is tracked over time to monitor the cardiac health of the patient. A 2-lead ECG may also be used. The R wave, of the ECG signal may be used for synchronization. Pulse oximetry may also be used.

The amplitude of cardiac oscillations is directly affected by pulmonary blood flow (PBF) in a linear manner, and the amplitude of this cardiac oscillation peak is likely correlated to the pulmonary blood volume variation (PBVV), which is defined as the change in the pulmonary blood volume from systole to diastole. PBVV has previously been investigated as a metric of cardiac function during heart failure. The PBVV reflects an increase in capillary volume that impinges upon the compliant bronchiole network leading to the alveoli of the lung and generates high frequency peaks in airway pressure during systole phase of the cardiac cycle. These peaks of cardiac oscillations can be detected. PBVV is proportional to the stroke volume and both values decrease as the cardiac output declines during heart failure. PBVV is also inversely proportional to increases in vascular resistance coincident with heart failure, which restrict the ability of the pulmonary capillaries to expand into the pulmonary airways and contribute to pulmonary hypertension. Thus, the standard deviation of cardiac oscillations (SDCOS) is directly proportional to cardiac output and inversely proportional to pulmonary artery pressure (PAP):

SDCOS∝a*(−ΔPAP)+b*ΔPBF

where a and b are constants representing compliance of the pulmonary arteries and bronchioles, respectively.

Pulmonary Arterial Compliance

Pulmonary Arterial Compliance (PAC) is related to Cardiac Heart Failure (CHF) and is a strong indicator of CHF. As the pulmonary artery becomes congested, PAP increases, as PAP increases, the pulmonary artery stretches. But, at higher pressures (above about 25 mmHg), the pulmonary artery becomes less able to stretch further which leads to increased pulse pressure within the pulmonary artery (pulmonary artery pulse pressure, or PAPP). As a result of the higher pressures within the pulmonary artery, more work is required from the right ventricle, and stroke volume (SV) is increased.

PAC can be Calculated as SV/PAPP (mL/mmHg)

Pulmonary arterial compliance has been shown to be a strong indicator of cardiovascular death or complications. As PAC decreases, the chance of cardiovascular complications or death increases. In addition, treatments for heart failure have been shown to increase the PAC. Currently, the only reliable way to measure PAC is with an invasive catheterization procedure.

Cardiogenic oscillations are generated by the cardiac pulsation in the pulmonary vasculature and are directly related to PAC. As heart failure worsens, stroke volume may decrease which leads to a decrease in the PAC amplitude. Also, PAP increases, the pulmonary artery stiffens, and PAPP increases, also leading to a decrease in the PAC amplitude. A decrease in PAC or PAC amplitude, is a strong indicator of worsening heart health. Amplitude in this instance refers to peak-to-peak amplitude of the curve.

In one use case example, the airway device/controller can be used to track a patient with congestive heart failure. If the patient using the airway device/controller is found to have decreased stoke volume or increased pulmonary artery pressure (via the pressure and/or flow sensors), decreased lung volume and/or decreased respiratory compliance due to fluid accumulation in the pleura and/or pulmonary spaces (via spirometer or pressure sensor) and/or enlargement of the heart, increased pathologic lung sounds (via the acoustic sensor/microphone), increased end-tidal CO2 (for example, after a breath hold) and/or an increased respiratory rate (via the pressure sensor or spirometer) then the healthcare provider or patient may be alerted that their condition is worsening.

In the home healthcare embodiment, the patient may then be sent home with a networked device (or return to the clinic) for repeat measurements. In the instance where this device is used in combination with daily weighings on a networked scale, the airway device/controller may communicate with an existing network provided by the scale or other in-home patient monitoring device, or any network, to alert the user and/or healthcare provider. In this way, the patient's cardiac health can be monitored remotely and noninvasively. This technique may also be used in lieu of radiographic examination to look for pneumo- or hemo-thorax following a procedure. Tension pneumothorax and detection of any other lung pathology may be accomplished with this technology, as well, in the hospital, office, or home setting.

In some embodiments, the airway device may be used to determine how to control fluid balance of a patient during dialysis. The amplitude of the COS signal is reduced when a patient is experiencing fluid overload, or dehydration. The controller can determine optimal patient hydration based on optimal COS signal amplitude.

In an alternative embodiment, the airway device/controller may record noises directly within the respiratory tract. In this embodiment, the airway device/controller may incorporate a disposable or reusable microphone attached to the airway device, (or alternatively, to a ventilator, vent tube or endotracheal tube). The microphone can track respiratory sounds and rapidly report the onset of respiratory distress, pneumonia, rales, rhonchi or other changes in lungs sounds. In its preferred embodiment the airway device/controller may incorporate noise cancellation functions. In one such embodiment, two microphones may be used within the airway device with one microphone facing the airway and the other microphone in a similar position within the airway device but sealed off from the airway. The signal from the sealed off microphone may then be subtracted from the microphone open to the airway thereby cancelling out ambient noise and allowing resolution of the physiologic sounds (cardiac, respiratory, gastrointestinal, etc.).

In some embodiments, the airway device/controller could be used in the placement and/or continuous monitoring of an endotracheal tube (ET). ET placement is related to causes of infection in ventilator-acquired pneumonia patients: poor placement can lead to pooling of fluid and, within the fluid, bacterial colonization can occur which then can migrate through the ET or around the cuff of the ET and into the lungs. Pooling of fluid and/or changes in respiratory flow/pressure can be monitored to obtain an early onset indication of infection. Bacteria may also be detected through sensors on the device.

In yet another embodiment, the airway device/controller can detect pathologic behavior of the heart valves. For example, when used in combination with an ECG, the expected mechanical heart behavior and timing of the cardiac cycle is known. By comparing the electrical and mechanical signals, improper mechanical function can be detected, such as the timing of the contraction of the atria or ventricles and opening or closing of the heart valves. Furthermore, the intensity and timing of these signals can also be used to diagnosis pathologies—for example, whether certain phases of the cardiac cycle are prolonged or incomplete, such as with mitral valve regurgitation. This information may be used alone or in combination with the sound information described above or with any other technique for diagnosing heart murmurs in order to better understand the underlying heart function or dysfunction.

This and any of the embodiments described herein may be utilized in a continuous or intermittent manner. The airway device may be designed to be worn by the user or require additional equipment to function and may be applied to the nose and/or mouth or applied directly to an endotracheal tube. The airway device/controller and any or all of its functions may be used in any setting including: the home, office, clinic, hospital ward, ambulatory surgery center (ASC) or intensive care unit (ICU)ca.

The airway device/controller may be used to monitor chronic conditions and/or detect acute conditions including: chronic obstructive pulmonary disease (COPD), asthma, CHF, cancer, stroke, pulmonary embolism, and any other condition that could have an impact on respiratory rate, temperature, stroke volume, heart rate, tidal volume, lung sounds, heart sounds, GI sounds, pO2, pCO2, pH, or any other of the monitored parameters.

The airway device may incorporate a controller to analyze the signals from the various sensors. Alternatively, all, or part, of a controller may exist separately from the airway device and communicate with the airway device either wirelessly (via internet, intranet, WAN, LAN or other network, or it may be local via Bluetooth, Wi-Fi, etc.) or wired. If the connection is wired, it may be continuous or intermittent. For example, the data from the airway device may be periodically transmitted via a USB connection or other type of connection after data has been collected. A wireless connection may also be continuous or intermittent. The controller may be, or communicate with, one or more mobile devices, computers, servers, etc.

One example of an embodiment of an apparatus for monitoring health may generally comprise a pressure sensor configured to sense a respiration pressure of a patient and at least one connector configured to be fluidly coupled with an assisted breathing device such that the pressure sensor is positionable to sense the respiration pressure from the patient. The apparatus may also include a controller in communication with the pressure sensor, wherein the controller is programmed to determine cardiogenic oscillation pressure measurements based on the respiration pressure sensed via the pressure sensor from the patient during a pause in airflow without logical integration with the assisted breathing device.

Another example of a method of monitoring health of a patient may generally comprise sensing a respiration pressure of a patient via a pressure sensor which is configured to be fluidly coupled to an assisted breathing device, and detecting or inducing a pause in airflow from the patient via the pressure sensor. The method may also include determining cardiogenic oscillation pressure measurements from the patient during the pause in airflow via a controller in communication with the pressure sensor and without logical integration with the assisted breathing device.

One variation of an apparatus for determining health of a user may generally comprise a housing configured to be held by the user when in use, a mouthpiece in communication with the housing and configured for insertion within a mouth of the user when in use, a temperature sensor positioned within or along the housing and configured for sensing a temperature of the user, a pulse oximeter sensor positioned along the housing for contacting the user and sensing a heart rate or blood oxygen level of the user, a microphone positioned within or along the housing for obtaining sounds associated with respiration from the user, and a controller configured to receive physiologic parameters including the temperature, heart rate, blood oxygen level, and sounds associated with respiration and determine a likelihood of contracting pneumonia, a presence of pneumonia, or a status of pneumonia within the user based on deviations from a threshold value which are present in at least two of the physiologic parameters.

One variation of a method of determining health of a user may generally comprise sensing a temperature of a user via a temperature sensor positioned within or along a housing, sensing a heart rate or blood oxygen level of the user via a pulse oximeter sensor positioned along the housing, sensing sounds associated with respiration of the user via a microphone positioned within or along the housing, receiving into a controller physiologic parameters including the temperature, heart rate, blood oxygen level, and sounds associated with respiration, and determining via the controller a likelihood of contracting pneumonia, a presence of pneumonia, or a status of pneumonia within the user based on deviations from a threshold value which are present in at least two of the physiologic parameters.

### BRIEF DESCRIPTION OF THE DRAWINGS

FIG. 1 shows one embodiment of the airway device/controller.

FIG. 2 shows an embodiment of the airway device/controller.

FIG. 3 is a graph showing an ECG overlaid on airway pressure data.

FIG. 4 is a graph showing pressure data from the ventilation tube of an animal.

FIG. 5 shows a graph of the ECG curve as well as corresponding cardiogenic oscillations waveforms.

FIG. 6 shows an embodiment of the airway device/controller

FIG. 7 shows an embodiment of the airway device/controller

FIG. 8 shows an embodiment of the airway device/controller

FIG. 9 shows an embodiment of the airway device used wirelessly with a controller in the form of a smart phone.

FIG. 10 shows an embodiment of the airway device connected to a controller in the form of a smart phone using a wired connection.

FIG. 11 is a block diagram of a data processing system, which may be used with any embodiments of the invention.

FIG. 12 shows an embodiment of a mouthpiece which includes a restrictor.

FIG. 13 shows an embodiment of a mouthpiece which incorporates a mechanical filter.

FIG. 14 shows an embodiment in which the restrictor and the sampling exit are combined

FIG. 15 shows an embodiment which incorporates a flow filter.

FIG. 16 shows a graph which demonstrates pulse pressure variability.

FIG. 17 shows an embodiment of the airway device/controller which includes a hand piece and at least some of the controller functions.

FIG. 18 shows another embodiment of the airway device/controller.

FIG. 19 shows several cardiogenic oscillation pressure curves and their average.

FIGS. 20A-C shows some alternative graphical displays.

FIGS. 21A-B show embodiments of the airway device/controller which include a hand-piece.

FIG. 22 shows an embodiment of the airway device/controller which has been incorporated into a continuous positive airway pressure (CPAP) device.

FIG. 23 shows an embodiment of the airway device/controller which may be standalone, or incorporated into a CPAP device.

FIG. 24 shows an embodiment of the airway device/controller which may be standalone, or incorporated into a CPAP device.

FIG. 25 shows a graph of the pressure signal from a rough pressure sensor, a sensitive differential pressure sensor and an ECG.

FIGS. 26A and 26B show 2 embodiments of the mouthpiece area of the airway device.

FIG. 27 shows a top view of an embodiment of the hand piece.

FIG. 28 shows a bottom view of an embodiment of the hand piece.

FIG. 29 shows how an embodiment of the airway device controller and how the system communicates data.

FIG. 30 shows examples of screens that may be shown on the display

FIG. 31 outlines a method of use of an embodiment of the airway device/controller device.

FIG. 32 shows an embodiment of the airway device which incorporates a spirometer, and other features.

FIGS. 33 and 34 show example screens of a training app.

FIGS. 35 and 36 show example screens of a survey.

FIG. 37 shows an embodiment of the airway device/controller being used in conjunction with a ventilator.

FIG. 38 shows the airway device attached in-line with the tubing or other component of a ventilator.

FIG. 39 shows the airway device attached externally to the tubing or other component of a ventilator.

FIG. 40 shows a graph of airway pressure and air flow over time within a typical ventilator system.

FIG. 41 shows a graph of airway pressure where an end inspiratory pause is not programmed to occur with every breath.

FIG. 42 shows several cycles of the airway pressure curve with periodic end inspiratory pauses.

FIG. 43 shows an embodiment of the airway device which effectively creates ventilator pauses.

FIG. 44 shows an embodiment of the airway pressure device which performs similarly to the device in FIG. 43.

FIG. 45A represents the pulmonary artery pressure curve of a healthy patient.

FIG. 45B represents the pulmonary artery pressure curve of a patient with pulmonary hypertension.

FIG. 46 shows a currently available capnography system.

FIG. 47 shows the airway device incorporated with a capnography system.

FIG. 48 shows a combination airway device/capnography system.

### DETAILED DESCRIPTION OF THE INVENTION

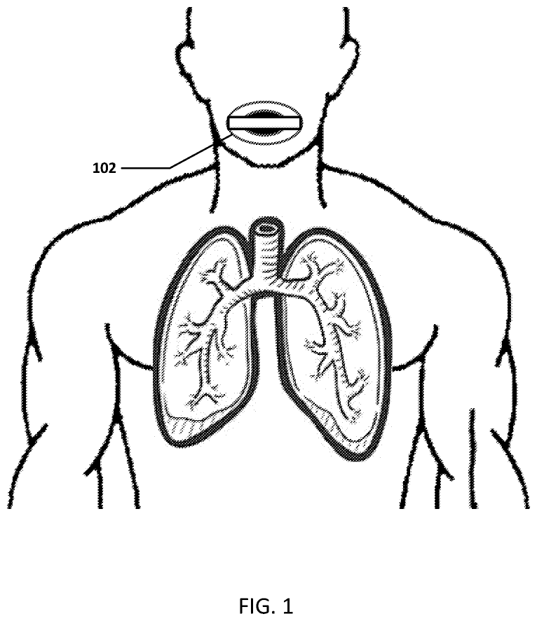

FIG. 1 shows an embodiment of airway device 102 worn in the mouth of a patient. One of the advantages of a portable embodiment, such as this one, is that it can be worn by a subject that is not only awake and not intubated, but upright and active. In other words, the use of the airway device is not limited to patients on a ventilator, CPAP, or other stationary medical device. The airway device/controller may be used on a patient/user with no additional ventilation support, or airway pressure support. Said another way, the airway device/controller may be used on a patient with or without a ventilator or CPAP machine or additional flow source, or any sort of artificial ventilation or airway pressure support. The airway device/controller may be used by patients/users who are breathing naturally or normally, or may be used in a “prompt mode”, where the controller prompts the user to do something other than breathe naturally. For example, the controller may prompt the user to hold his/her breath, hold his/her breath after inhalation, hold his/her breath after exhalation, hold his/her breath “now”, perform the MVM at a given pressure for a certain period of time, etc.

The airway device contains one or more sensors which can measure and/or calculate airway pressure, airway flow, temperature, sounds, respiratory rate, stroke volume, heart rate, tidal volume, lung sounds, heart sounds, GI sounds, pO2, pCO2, pH, ECG, pulse rate, pulse pressure, spirometry, analytes and/or compounds in the breath (i.e. urea, markers of infection, O2, CO2, urea, water vapor, alcohol, drugs, etc.) or analytes and/or compounds in the saliva, such as glucose, etc.

A controller is either incorporated into the airway device or a separate device which communicates with the airway device either wirelessly or via a wired connection. The controller may be incorporated into a ventilator, a CPAP, an assisted ventilation device, a stand-alone device or incorporated into, or in communication with, a computer and/or smartphone.

In a preferred embodiment, the controller is incorporated into a smartphone which communicates wirelessly with the airway device, either on a continuous or intermittent basis. Data transferred from the controller may also be transmitted to/from a remote server, for example, via the internet or an intranet. Data from the controller may also be anonymized. Anonymized data may be aggregated across patients for trends analysis. Data collected may include metadata such as patient ID, timestamp, patient medical history, such as weight, medications, etc. Use of the term “airway device” herein may include a controller component.

The airway device may have a portion within the mouth or be completely external. It may also be over the nose either instead of, or in addition to, the mouth. The airway device may purposefully block the nose. The airway device may also be incorporated into an endotracheal tube.

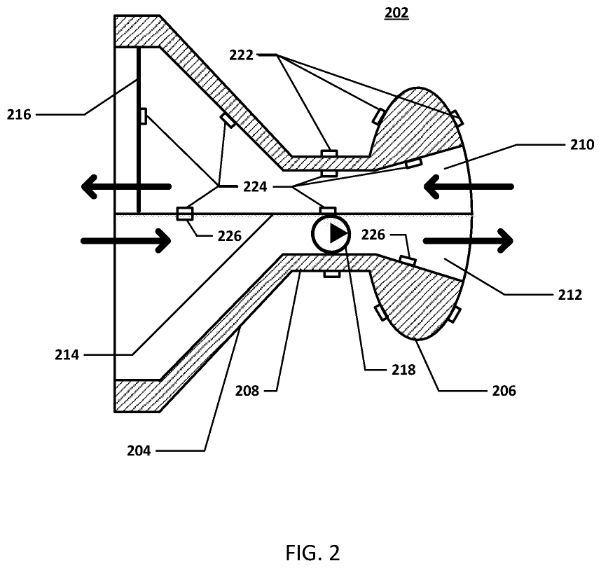

FIG. 2 shows a detailed view of an embodiment of airway device 202. This embodiment includes external opening section 204, mouthpiece section 206 and neck section 208. The mouthpiece device in this embodiment includes one or two airway lumens, exhalation airway lumen 210 and inhalation airway lumen 212. In this embodiment, the two lumens are separated by divider 214. Alternatively, only one lumen may be present, for example, only an exhalation lumen.

Gas outflow vent 216, in the exhalation airway lumen, may include a spirometry function. The vent may also maintain or cause to be maintained a slight positive pressure so that the airway of the subject remains open during breathing, which aids in the ability to sense certain parameters.

The air inflow, or inhalation airway lumen, and/or the exhalation airway lumen, may include one-way 218 valve to help direct exhaled air through the exhalation airway lumen during breathing.

Sensors 222, 224, and 226 may sense any of the parameters listed herewithin. Sensors may be placed in the exhalation airway lumen 210, the inhalation airway lumen 212, or on the outside of the airway device. Sensors 222 on the outside of the device will generally be for contact sensing with the mucosa and/or the lips, such as ECG sensors. Sensors 224 in the exhalation airway lumen may measure parameters associated with exhaled air, including pressure, flow, sounds, O2, CO2, urea, water vapor, alcohol, drugs, etc. Sensors 226 in the inhalation airway lumen may measure parameters associated with inhaled air, including O2, CO2, urea, water vapor, alcohol, drugs, etc.

Generally, the sensors can be placed anywhere along the length of the airway device, but there may be advantages to certain locations for certain types of sensors. For example, sensors for temperature, water vapor, alcohol, drugs etc. measured in exhaled air, would likely be better placed closer to the subject.

Flow and/or pressure sensors can be placed anywhere along the length of the airway device, but there may be an advantage to placing these sensors in a narrow and/or constant diameter section of the airway device such as within neck 208. A sensor or sensors may also be placed on gas outflow vent 216. Sensors may also be remote. For example, a pressure sensor, for example a pressure transducer, may be in fluid communication with the mouthpiece via a tube with an inner lumen.

A single use barrier may be used to cover mouthpiece section 206 to maintain sterility of the airway device. Alternatively, a disposable mouthpiece section may be attached to the airway device and removed after use. A heat-moisture exchanger may be used to prevent humidity from the breath entering into the device. Alternatively, the airway device may be sterilizeable or disposable.

Airway device 202 may incorporate hardware and/or software to either act as a controller, or communicate with a controller. The airway device may also act as a “partial controller”, where some of the controller activities take place within the airway device, and some take place within a separate controller device.

Airway device 202 may be made out of any suitable material or materials, including polymer, metal, or any other material or any combination of materials. Airway device is preferably relatively light and portable. The airway device may be powered by one or more batteries.

Flow/pressure sensors may include orifice plates, pressure transducers, cone devices, Pitot tubes, Venturi tubes, flow nozzles, Fleisch or Lilly type pneumotachometers, or any other suitable technology. Sensor resolution is generally high. Pressure sensor sensitivity may around +/−0.5 mmHg. Pressure sensor sensitivity may around +/−1 mmHg. Pressure sensor sensitivity may around +/−2 mmHg. Alternatively, pressure sensor sensitivity may around +/−10 mmHg. Alternatively, pressure sensor sensitivity may around +/−20 mmHg.

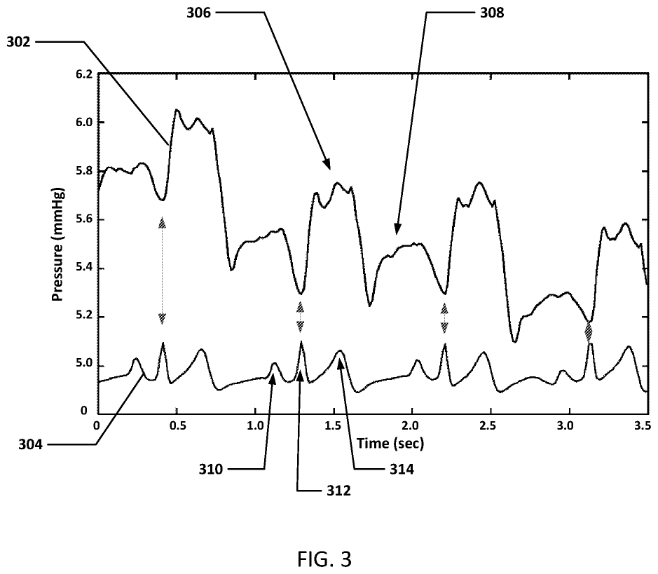

FIG. 3 shows a graph of an ECG along with simultaneously measured airway pressure data. ECG data 304 is shown below airway pressure data 302. Within the airway pressure, systolic pulse data 306 and diastolic pulse data 308 are clearly visible. Within the 3-lead ECG data, P wave 310, QRS complex 312, and T wave 314 are all visible. The double headed arrow lines show where the QRS complex peak lines up with the valleys of the pressure data.

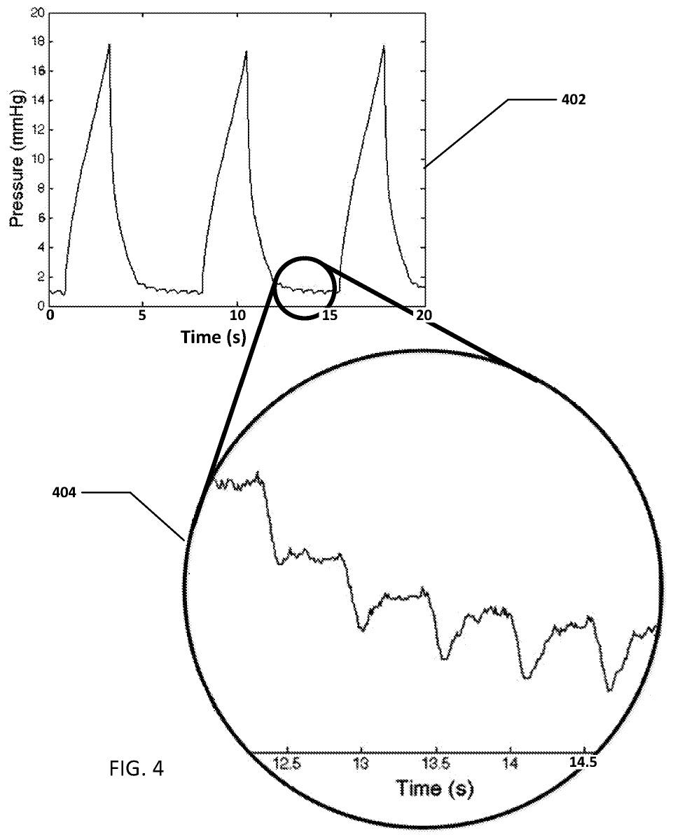

FIG. 4 shows a detailed view of the pressure data between respirations shown in graph 402. Cardiogenic oscillations can be seen in detailed view 404 of pressure vs. time. The amplitude or area under the curve for these pulses can be used as an indicator of relative cardiac output and/or pulmonary artery pressure. Not shown but also useful in the same manner are cardiogenic oscillations in the flow signal.

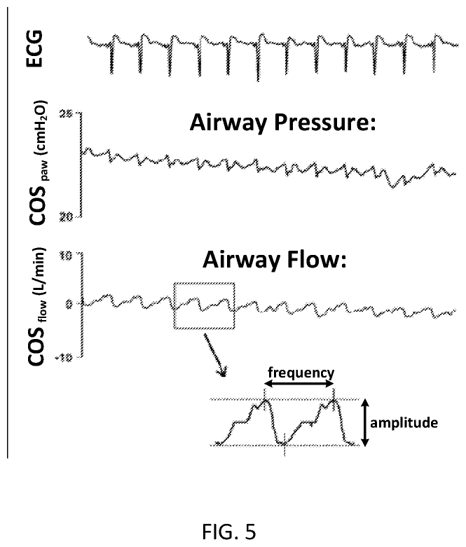

FIG. 5 shows a graph of the ECG curve, the cardiogenic oscillations waveform generated using data from pressure sensor(s), and the cardiogenic oscillations waveform generated using data from flow sensor(s). Also shown are the amplitude and the frequency of a cardiogenic oscillations waveform.

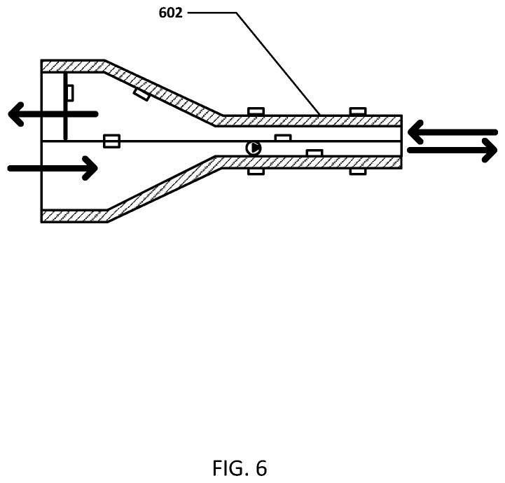

FIG. 6 shows another embodiment of the airway device. The neck portion 602 is extended so that it also serves as the mouthpiece portion, which is more straw-like than the previously shown embodiment.

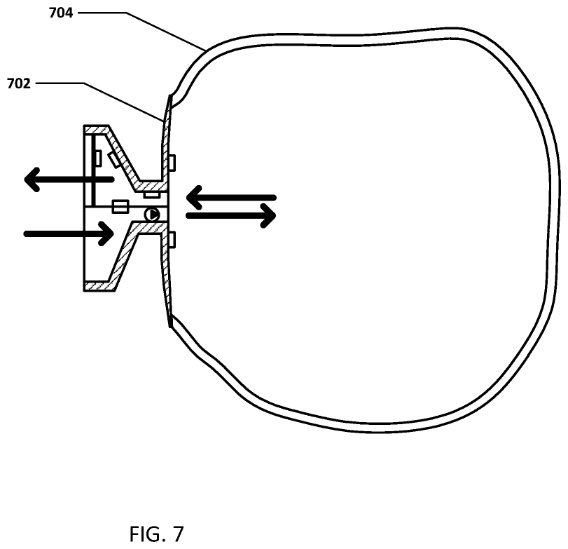

FIG. 7 shows another embodiment of the airway device. Mouthpiece area 702 is flat and designed to go over the lips/mouth. Strap 704 may hold the device on the face of the subject.

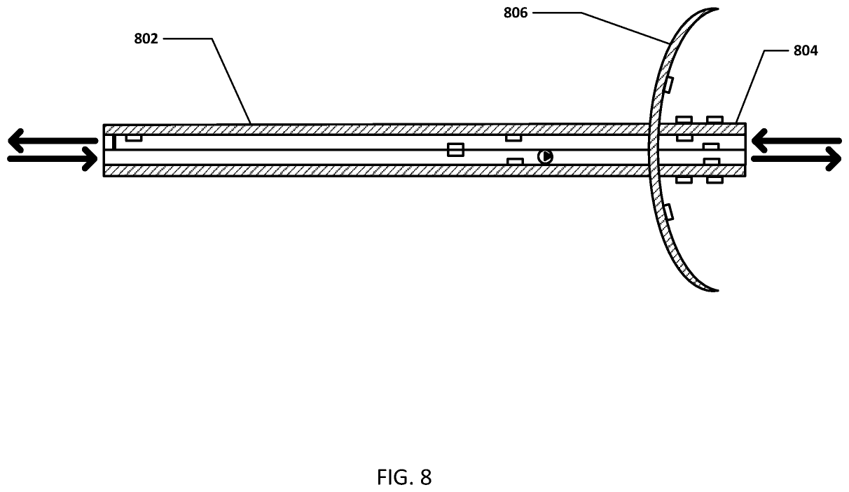

FIG. 8 shows another embodiment of the airway device. External opening section 802 of this embodiment is elongated and more narrow than previously shown embodiments. Section 802 may be flexible, as in flexible tubing, or may be rigid, or may be partially flexible and partially rigid. Mouthpiece section 804 may include mouth shield 806 to help keep the device in place. The various sensors and/or valves may be anywhere along the length of this embodiment.

FIG. 9 shows an embodiment of the airway device and controller where the controller is separate from, and may be remote to, at least in part, the airway device. In this embodiment, controller 904 is a smart phone and communicates wirelessly with airway device 902, which may include a wireless data transmitter.

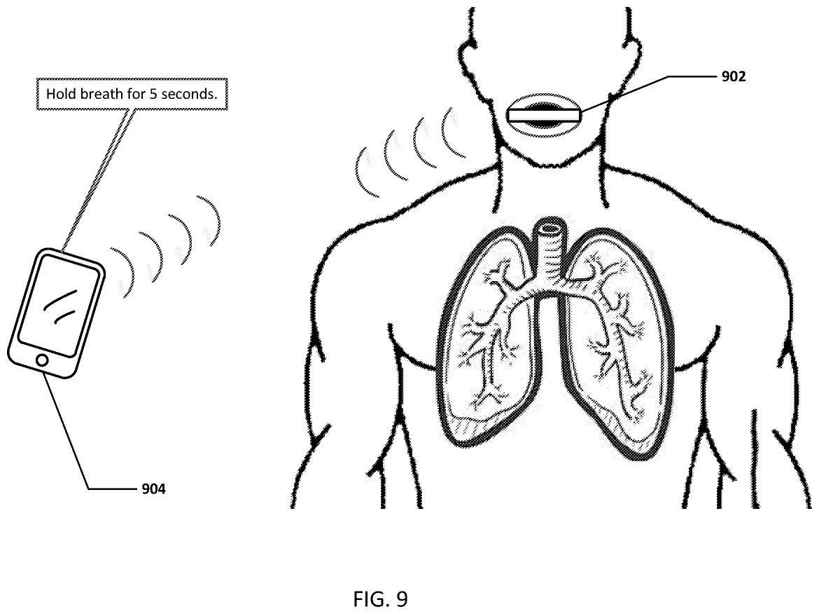

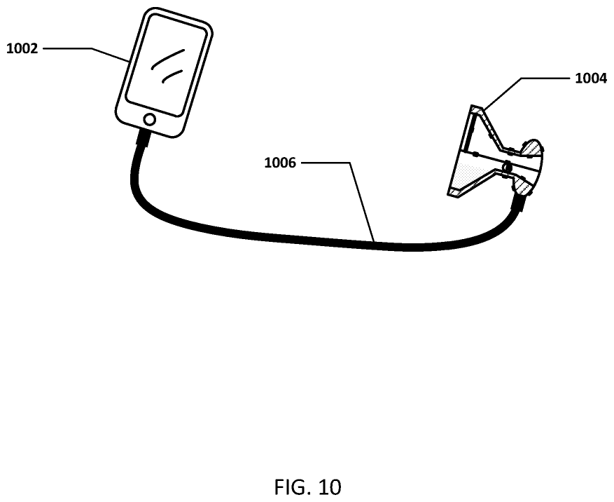

FIG. 10 shows an embodiment of the airway device and controller where the controller is separate, at least in part, from the airway device. In this embodiment, controller 1002 is a smart phone and communicates with airway device 1004 via a “wire” or cable, for example, a USB cable. In this embodiment data may be collected and stored in airway device 1004 and periodically uploaded to controller 1002 via cable 1006. Alternatively, data may be stored, at least in part, in the controller.

The controller, whether it is separate from the airway device, or incorporated into the airway device, or some functions are located in the airway device and some located separately, may function as follows. The controller collects the data from the various sensors and analyzes them to determine cardiac output, stroke volume and/or cardiac function and/or other parameters. In addition, the controller may prompt the subject to help obtain the data from the sensors. For example, the controller may prompt the subject to hold his/her breath. The breath holding prompt may happen at certain phases of the breathing cycle, such as before or after inhalation and/or exhalation. The controller may prompt the subject to breath at a certain rate or to inhale, exhale, or hold his/her breath for a certain time period, or within a certain goal pressure range. Indicators may be present on the controller and/or the airway device to help the subject time certain activities, or achieve certain breathing goals, such as exhale pressure. For example, the controller may prompt the subject to hold his/her breath until a light on the controller and/or airway device turns green, or until an auditory signal is heard.

The controller may also determine whether the data it is collecting is adequate for analysis. For example, if the subject's airway is closing between breaths, or during exhalation, the data may be more difficult to analyze. The controller can sense when this is happening either by the pressure/flow profile or other parameters and can prompt the subject to adjust his/her breathing. For example, the controller may prompt the subject to breath more slowly, or to sit still. In addition, the controller may change the positive pressure of the airway device to help keep the airway open. Some possible prompts that the controller may provide to the subject are:
hold your breath for x seconds
hold your breath until the indicator does x
Breath normally until the indicator does x
exhale at a consistent pressure as shown on indicator
exhale at a consistent pressure as shown on indicator for x seconds (or until indicator says x seconds has elapsed)
exhale with throat open at a consistent pressure as shown on indicator for x seconds
perform the MVM for x seconds
exhale and then hold breath
inhale and then hold breath
breath normally
breath more slowly
Breath more quickly
Breath in slowly
Breath out slowly
Breath in quickly
Breath out quickly
testing is complete
begin exercising
end exercise

Other prompts are also possible. The prompts may change depending on the data being collected. For example, if the controller determines that the airway is closing between breaths, during breathing, or during exhalation, the prompts may tell the subject to breathe differently, or the controller may cause the airway device to apply positive pressure to the airway. In addition, the user may be prompted at certain time(s) of the day to use the device, so that the device is used at the same time each day. For example, the device may prompt the user to use the device upon waking.

Other parameters that may be considered in determining whether the subject's breathing is optimal for data collection include: variability of peak-to-peak period and magnitude, waveform shape, etc.

The controller may analyze the data from the sensors to determine other conditions, including COPD, asthma, CHF, cancer, stroke, pulmonary embolism, dyspnea, paroxysmal, nocturnal dyspnea, emphysema, and any other condition that could have an impact on respiratory rate, temperature, stroke volume, heart rate, tidal volume, lung sounds, heart sounds, GI sounds, pO2, pCO2, pH, alcohol, urea, drugs, or any other of the monitored parameters.

Vagal tone/vasovagal syndrome may also be determined using the present invention. Slight changes in heart beat parameters, including amplitude, rate, waveform shape, etc., at different stages of the breathing cycle can be measured and vagal tone determined. For example, if the heart rate increases during inhalation, this may indicate a high vagal tone.

Example of Data Processing System

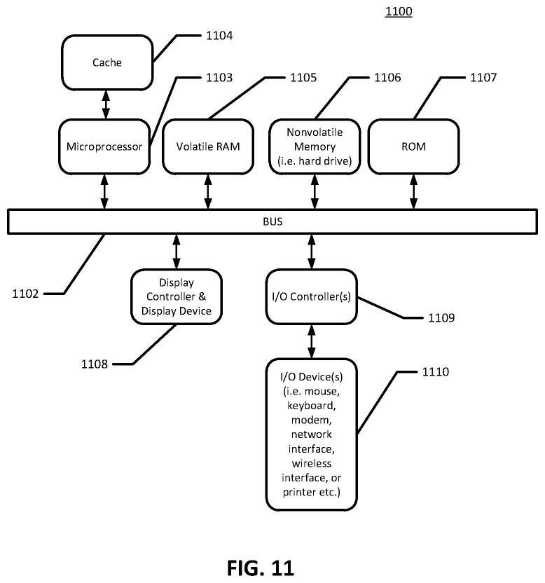

FIG. 11 is a block diagram of a data processing system, which may be used with any embodiment of the invention. For example, the system 1100 may be used as part of a controller, server, mobile device, hand piece, computer, tablet, etc. Note that while FIG. 11 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 11, the computer system 1100, which is a form of a data processing system, includes a bus or interconnect 1102 which is coupled to one or more microprocessors 1103 and a ROM 1107, a volatile RAM 1105, and a non-volatile memory 1106. The microprocessor 1103 is coupled to cache memory 1104. The bus 1102 interconnects these various components together and also interconnects these components 1103, 1107, 1105, and 1106 to a display controller and display device 1108, as well as to input/output (I/O) devices 1110, which may be mice, keyboards, modems, network interfaces, printers, and other devices which are well-known in the art.

Typically, the input/output devices 1110 are coupled to the system through input/output controllers 1109. The volatile RAM 1105 is typically implemented as dynamic RAM (DRAM) which requires power continuously in order to refresh or maintain the data in the memory. The non-volatile memory 1106 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 11 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1102 may include one or more buses connected to each other through various bridges, controllers, and/or adapters, as is well-known in the art. In one embodiment, the I/O controller 1109 includes a USB (Universal Serial Bus) adapter for controlling USB peripherals. Alternatively, I/O controller 1109 may include an IEEE-1394 adapter, also known as FireWire adapter, for controlling FireWire devices.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the figures herein may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

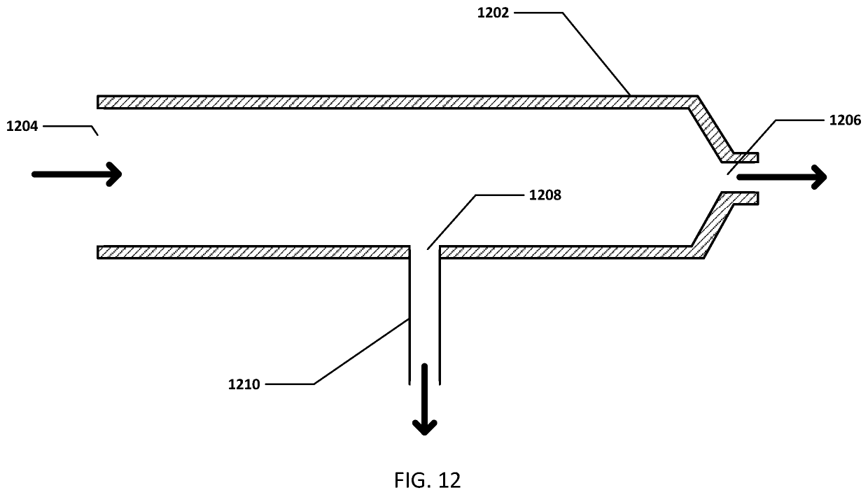

FIG. 12 shows an embodiment of an airway device which includes a restrictor. The restrictor helps reduce turbulent air flow within the airway device. Airway device 1202 in this embodiment has mouth opening 1204, which is larger than restrictor 1206. Restrictor 1206 is open to ambient air. As the user exhales into the airway device, restrictor 1206 restricts the airflow which increases the laminar nature of the air flow within the airway device. In this embodiment, as the user breathes through opening 1204, some air exits restrictor 1206, however some air, preferably air which is predominantly flowing in a laminar manner, exits sampling exit or lumen 1208. Sampling exit 1208 may connect directly to a pressure, or other, sensor, or it may connect to a pressure sensor or other sensor via connector 1210, which may be a flexible or rigid tube. The purpose of restrictor 1206 is to reduce turbulence in the air flow within the airway device so that the air exiting sampling exit 1208 is as laminar as possible. Note that this figure is showing an exhalation lumen only. A separate inhalation lumen may be incorporated into the device and/or the subject may be asked to inhale separately, either through his/her nose, or by removing the device from his/her mouth. Alternatively, the patient may also use the exhalation lumen for inhalation.

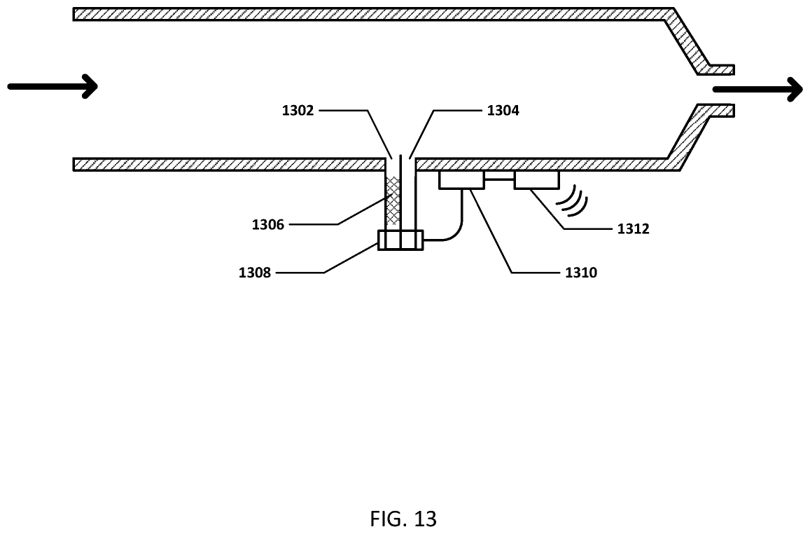

FIG. 13 shows an embodiment of an airway device which incorporates a mechanical filter. In this embodiment there are at least two sampling lumens, 1302 and 1304. One of the sampling lumen includes mechanical high pass filter 1306. The pressure sensor in this embodiment is a differential pressure sensor. Differential pressure sensor 1308 is in fluid communication with at least two sampling lumens or inputs, and compares the pressure reading between the two lumens. This configuration produces a cleaner pressure signal for analysis by circuit board 1310 by filtering out the pressure from the breaths and leaving those from the cardiogenic oscillations. Circuit board 1310 may be incorporated into the airway device or may be separate, for example on a separate controller, and communicated with either wirelessly or via wire. In this embodiment, the circuit board is incorporated into the airway device and communicates with a controller via wireless transmitter 1312. In this embodiment, circuit board 1310 and wireless transmitter 1312 may be considered to be part of the controller as well, for purposes of defining the controller. Filter 1306 may be made out of any suitable material including foam or any membrane that is semi-permeable to air. Note that this figure is showing an exhalation lumen only. A separate inhalation lumen may be incorporated into the device and/or the subject may be asked to inhale separately, either through his/her nose, or by removing the device from his/her mouth. Alternatively, the patient may also use the exhalation lumen for inhalation.

The mechanical high-pass filter isolates the higher frequency cardiac oscillation signal from the lower frequency pressure signal associated with natural breathing. This filter may employ a partially-impermeable barrier between differential sensing and reference inputs. The high-frequency cardiac oscillation signal is seen by the sensing input, whereas the pressure changes due to breathing are low frequency enough to equilibrate across the membrane and are detected at both inputs. By breathing into the device with a slight expiratory pause, or using the MVM, the cardiogenic oscillation signal can be reliably captured. Some embodiments may incorporate an additional, less sensitive, pressure sensor to monitor the entire breathing cycle and provide feedback to the patient about the size and frequency of the breaths, improving repeatability between measurements.

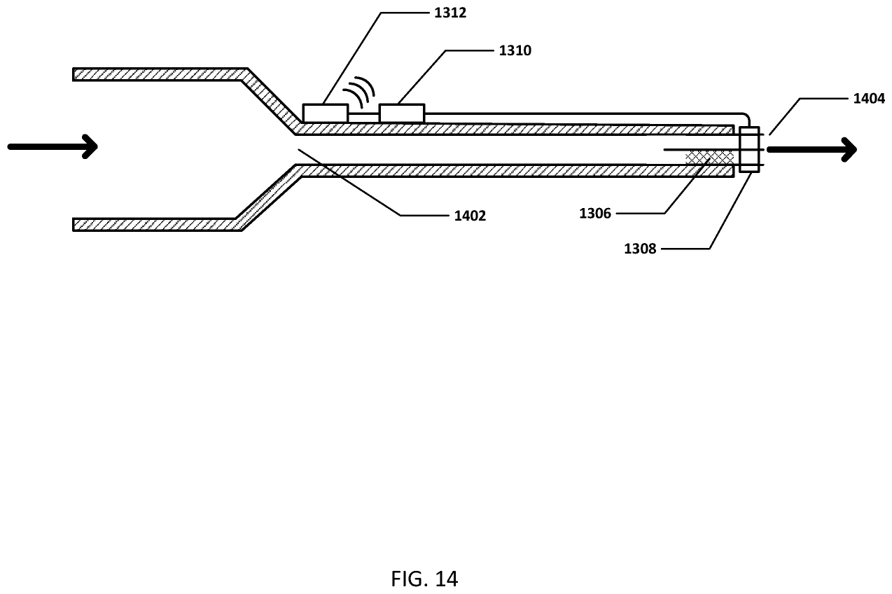

FIG. 14 shows an embodiment in which the restrictor and the sampling exit are combined. Restrictor 1402 reduces the turbulence in the airflow as air is breathed in and out of the airway device. Breathed air exits and may enter via outlet 1404. Differential pressure sensor 1308 may allow air to flow through it or alongside it to exit the airway device, or alternatively, the airway device may have an additional air exit (not shown). Note that this figure is showing an exhalation lumen only. A separate inhalation lumen may be incorporated into the device and/or the subject may be asked to inhale separately, either through his/her nose, or by removing the device from his/her mouth.

Note that the restrictor could be anything suitable, such as a flow control valve, a pressure control valve, etc.

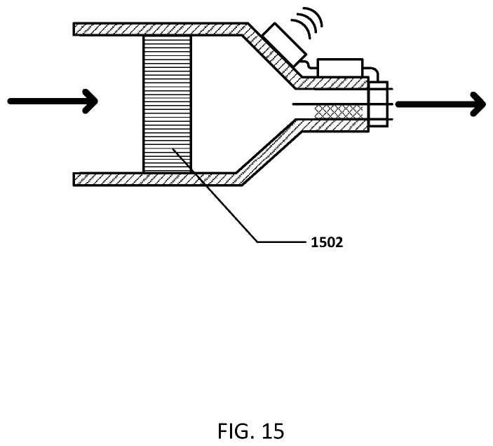

FIG. 15 shows an embodiment which incorporates a flow filter. Flow filter 1502 decreases the turbulence of the airflow coming into the airway device. In this embodiment, flow filter 1502 is used instead of a restrictor. The airway device may have an additional air exit (not shown). Flow filter 1502 may be made out of any suitable material such as polymer and in any suitable configuration such as a honeycomb or parallel capillary configuration. Note that this figure is showing an exhalation lumen only. A separate inhalation lumen may be incorporated into the device and/or the subject may be asked to inhale separately, either through his/her nose, or by removing the device from his/her mouth.

Any of the embodiments herein can be adapted to be used inside the mouth, or partially inside the mouth. For example, an airway device deeper inside the mouth may be advantageous in keeping the airway open for cleaner pressure measurements. Furthermore, any of the embodiments herein may also be adapted to be used with patients who are tracheally intubated, in which case the devices described are attached to or in-line with the tracheal tube.

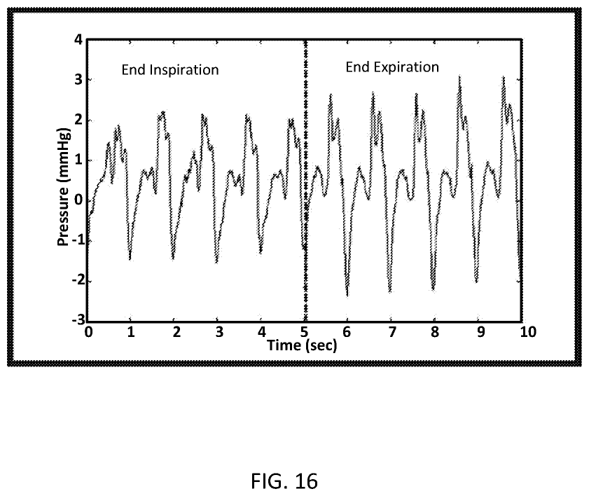

FIG. 16 shows a graph which demonstrates pulse pressure variability. As mentioned earlier, variability in the respiratory pulse pressure waveform can be used to determine hydration status, as well as volume status, and also pulmonary artery compliance. The graph in FIG. 16 shows the pulse pressure at end inspiration and at end expiration. Pulse pressure is defined as the difference between the systolic and diastolic pressure readings, or the amplitude of the waveform (lowest point to highest point). The difference in amplitude between these two waveforms is the pulse pressure variability. A large variability may indicate dehydration, where a decrease in variability over time may be an indicator that hydration is being restored or has been restored.

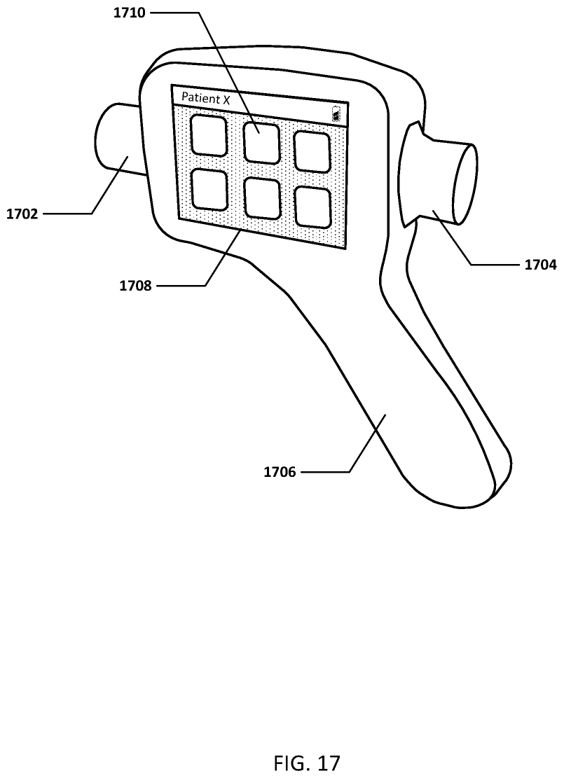

FIG. 17 shows an embodiment of the airway device/controller which includes a hand piece and at least some of the controller functions. The airway device of this embodiment includes 2 mouthpieces 1702 and 1704. The user breaths into one of these mouthpieces and breath exits through the other mouthpiece. Hand piece 1706 is held by the user or by the user's physician. Display 1708 displays one or more display areas 1710. These display areas may include buttons, or links, to more information, such as settings, waveforms, including waveforms showing HR (heart rate), SV (stroke volume), CO (cardiac output), PAC (pulmonary arterial compliance, etc., analytical results of waveform analysis, triggers for alarms/notices, etc. The airway device/controller of this embodiment may communicate wirelessly, or in a wired manner with one or more mobile devices, computers, servers, etc.

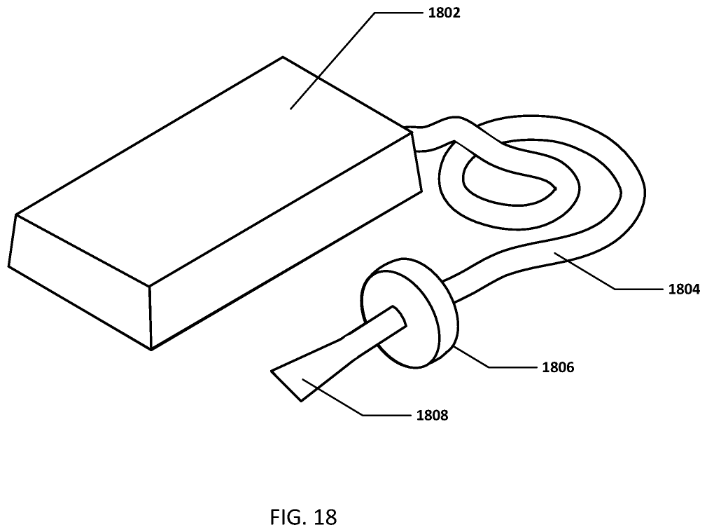

FIG. 18 shows another embodiment of the airway device/controller. This embodiment includes controller 1802, signal transmission tubing 1804, heat-moisture exchanger/filter 1806 and mouthpiece 1808. In this embodiment, the pressure/flow sensor may be in the controller. In use, during user breathing into the mouthpiece, the pressure in the airway is transmitted to the pressure/flow sensor via mouthpiece 1808 and tubing 1804. Note that controller 1802 may be in communication with a computer/mobile device/networked server etc., which may include some of the controller functions.

Embodiments of the airway device/controller may also be incorporated with a standard or specialized inhaler, for example for asthma. The airway device/controller in these embodiments may include a feature which tracks usage of the airway device and/or inhaler to monitor use compliance.

Embodiments of the airway device/controller may include integration with electronic health records (EMR) or electronic health records or other systems. For example, data from the controller may be transmitted wirelessly (or wired) to a server in the internet which integrates the data with that of an EMR. The patient ID (possibly anonymized) may be integrated into the metadata of the data transmitted by the controller so that the data can be integrated with the correct patient's medical record.

Data from multiple airway devices/controllers may be collected and aggregated and analyzed for trends. This data may be anonymized to comply with privacy rules.

In some embodiments of the airway device/controller, respiratory sinus arrhythmias (changes in heart rate due to breathing) may be tracked as an indicator of heart health or heart failure. Deviations from trends may be indicative of heart failure issues and may provide an alert. Because the data collected by the airway device may be continuous, for example, while the user sleeps, deviations from the norm (either for that patient or for a patient population) may indicate changes in health, and in particular, heart health.

In some embodiments of the airway device/controller, the device is used in an ambulatory manner. In other words, the user may use the device while walking around, watching TV, working, sleeping, resting, exercising or while performing everyday activities. The user is not tied to a stationary device, hospital nor clinic.

Sensors connected to the airway device/controller may include a blood oxygen saturation sensor or a blood CO2 saturation sensor or any other type of oxygen/CO2 sensor. For example, blood oxygen saturation may be determined by a pulse oximetry sensor in contact with the lips, tongue, oral mucosa and/or finger/extremities. This signal and/or an EKG signal collected from one or more EKG sensors (which may be in contact with these, or other, locations) may be used to determine pulse transit time.

Tissue O2/CO2 may be determined using an air tonometry sensor in contact with, or in proximity to, the tongue or oral mucosa or elsewhere. This type of sensor may include an air permeable membrane between the sensor and the body.

Absolute stroke volume may also be determined as follows. The volume or air displaced by each cardiac contraction due to pressure changes is determined by first determining the volume of air in the lungs. This may be done in one or more of several ways:

1) pulsed air method—A known volume of air is pulsed into the lungs and the change in pressure is measured. From this, the volume of dead space in the lungs may be determined.

2) spirometry—Total lung volume can be estimated from spirometry.

3) gas dilution—A known quantity and concentration of a target gas is infused into the lungs. The concentration of the target gas is then measured in the exhaled air exhaled to determine how much air has mixed with the target gas, thus providing an estimate of lung volume.

Stroke volume variability may also be determined/calculated. For example, the controller may prompt the user to breathe in deeply. The controller may use data captured from a sensor/sensors to determine stroke volume measurements at end inhalation and at end expiration—potentially to determine stroke volume variability. The controller may correct for changes in cardiac pulse size due to change in lung volume using spirometry (which measures breath volume) or the pulsed air method, or gas dilution techniques.

Spirometry may be used to measure one or more of several parameters, including: Vital capacity (VC), Forced vital capacity (FVC), Forced expiratory volume (FEV) at timed intervals of 0.5, 1.0 (FEV1), 2.0, 3.0 seconds, and other intervals, forced expiratory flow 25-75% (FEF 25-75), maximal voluntary ventilation (MVV), also known as Maximum breathing capacity, Peak Expiratory Flow (PEF), and any other parameters. Other tests may be performed. Results may be provided in raw data (liters, liters per second) and percent predicted—the test result as a percent of the “predicted values” for the patients of similar characteristics (height, age, sex, and sometimes race and weight), or the results may be provided in other ways.

In some embodiments of the airway device/controller, an ECG signal of the user is collected simultaneously to the cardiogenic oscillation data. In this way, the precise length and/or timing of a heartbeat can be determined (by the ECG signal) and the cardiogenic oscillation pressure curve can be divided up into precise heartbeats. In other words, one or more cardiogenic oscillation curves, each relating to one heartbeat, can be collected and identified and averaged, because the start and end of the cardiogenic oscillation curve relating to each heartbeat is precisely identified by the ECG signal. This allows collecting more than one cardiogenic oscillation curve and averaging them to get more accurate cardiogenic oscillation curve data. One or more ECG sensor/electrode(s) may be placed on the mouthpiece, or handheld portion of the airway device. ECG sensor/electrode(s) may be in contact with the user's mouth, finger(s), hand(s), or elsewhere on the body. Various features of the ECG curve may be used to “gate” the cardiogenic oscillation pressure curve. For example, the R peak, or alternatively the P, Q, S, T, U areas may be used.

Alternatively, or in addition, the signal from a pulse oximeter/photoplethysmograph may be used to gate the cardiogenic oscillation pressure curve in the same way—to determine the precise length/timing of the heartbeat. The same way a feature of the ECG curve can be used as a gating feature (for example, using the time between subsequent peaks of the R-wave), a feature, peak, valley, slope, length etc. of the pulse oximeter curve may be used instead of, or in addition to, the ECG curve to gate the pressure curve data. Multiple ECG and/or multiple pulse oximeter signals may alternatively be used. For example, the device may have electrodes/sensors for pulse oximeter and ECG for each hand, resulting in 2 ECG signals in addition to 2 pulse oximeter signals. This allows the best signal to be used to gate the cardiogenic oscillation pressure curve. The best signal may be chosen by amplitude, identifiable peak, consistency, etc. In this way, a good signal is likely to be obtained even if the user is not touching, or in contact with, all of the electrodes/sensors perfectly. Where redundant sensors are used to gate the cardiogenic oscillation pressure curve, the redundant sensors may be set with different gains on each. In this way, if one of the signals maxes out, or rails, where the peak of the curve is difficult to identify, another signal may be lower and have more identifiable peaks. This situation may occur if a user is pressing the sensors with a lot of pressure. In this way, one device with different sensors set with differing gains, may accommodate users with different finger pressures.

Outliers, or less useful data, may also be removed using ECG and/or pulse oximetry signals from the analysis to optimize the analysis results. More than one collected ECG/pulse oximetry signal may also be used in the analysis. One or more pulse oximeter/photoplethysmograph sensor/electrode(s) may be placed on the mouthpiece, or handheld portion of the airway device. Pulse oximeter/photoplethysmograph sensor/electrode(s) may be in contact with the user's mouth, finger(s), hand(s), or elsewhere on the body.

Alternatively, or in addition, the signal from the rough pressure sensor may be used to gate the cardiogenic pressure curve in the same way—to determine the precise length/timing of the heartbeat. Any sensor that determines and communicates the length/timing of a heartbeat to the controller may be used to gate the cardiogenic oscillation pressure curve. In some embodiments, the cardiogenic oscillation pressure curve is used to gate itself using features of the cardiogenic oscillation pressure curve.

In some embodiments, the gating may be performed using any of the methods identified herein, and in addition, the controller may analyze the COS or gating curves for fibrillation, such as atrial fibrillation. The controller may not include curves which are affected by fibrillation in the analysis of the gating length.

In any of the gating curves, a regularly repeating feature, or peak, of the curve may be used to assess the quality or relative quality of the gating curve to be used as gating, as well as for the gating itself.

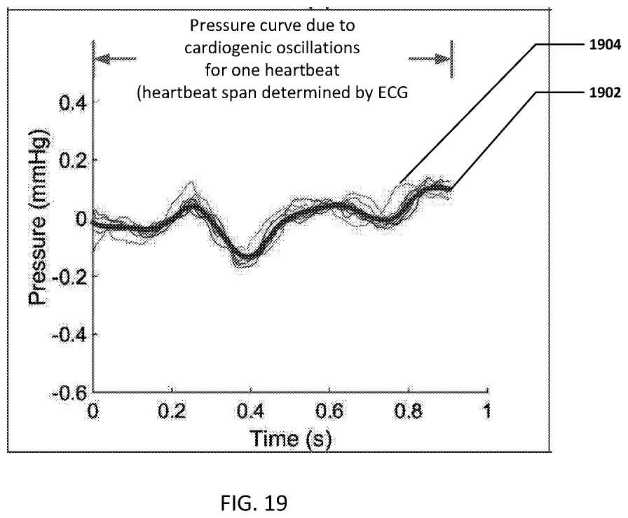

FIG. 19 shows several gated cardiogenic oscillation pressure curves layered on top of one another, along with the average of the curves. The thinner lines 1902 represent the individual cardiogenic oscillation curves obtained from several heartbeats. The heartbeats are separated on layered on top of each other and the average curve 1904 is calculated by the controller. The beginning and end point for each heartbeat can vary slightly over time making it difficult to average the curves without a way of precisely identifying the start and end point of each heartbeat (i.e., gating). Here, the controller does this by using the simultaneously obtained ECG signal from the patient. By picking one or more points in the ECG, for example, the R peak, the beginning and end of each heartbeat can be precisely identified and as a result, cardiogenic oscillation curves from multiple heartbeats can be averaged. The controller may also be equipped to determine the best point in the ECG to use for the heartbeat identification, for example the ECG curve may be analyzed for the most consistent area/peak, the most identifiable area/peak, the sharpest peak/area etc.

In the analysis shown in FIG. 19, the median curve may be used instead of, or in combination with, the mean curve. To construct the median curve, the median value at each point in time (for example, at 1, 2, 3, . . . ms after detection of the R peak) is calculated. This may help to reduce the influence of individual outlier curves, although other techniques may be used to accomplish the same goal, such as excluding the points that fall outside a specified window of deviation when calculating the mean.

Analysis of the shape of the cardiogenic oscillation curve may provide information on the patient. It is also patient specific, meaning that even among healthy patients, the cardiogenic oscillation curve shape is unique to each individual. In this way, the shape of the curve can be used as a signature to identify a patient. Changes in the cardiogenic oscillation curve may indicate specific disease states, or relative disease states. For example, the shape of the curve (or the change in the shape of the curve compared to normal or over time) may be analyzed to determine, and/or track over time, any of the following disease states.

Aortic stenosis

Aortic insufficiency

Mitral stenosis

Mitral insufficiency (regurgitation)

Pulmonary stenosis

Tricuspid stenosis

Tricuspid regurgitation

Pulmonary hypertension

Pulmonary fibrosis

congestive heart failure

Acute respiratory distress syndrome

Ventilator-acquired pneumonia

Pneumonia

Atrial Septal Defect

Patent Foramen Ovale

Single ventricle

Others

In addition, changes to the shape of the cardiogenic oscillation curve over time, or with the patient in different positions, may indicate other patient parameters such as hydration. For example, a cardiogenic oscillation curve which appears to show improvement in PAC when the patient's legs are raised may be an indicator of dehydration. Hydration status may also be evaluated by changing the patient's position and/or breathing pressure. For example, the user may be prompted by the controller to take a measurement while standing, supine, sitting, legs raised, at a specific angle, inverted, etc. The relationship between or among these readings may be used in the data analysis to determine patient health. For example, the ratio between supine COS data and sitting COS data may be used in the analysis.

In some embodiments, any cardiovascular and/or pulmonary parameter may be collected in more than one patient position and the relationship between the parameters used to determine the health of the patient. For example, other available devices, such as the CardioMEMS™ device, manufactured by St. Jude Medical, may be used to collect a patient parameter, such as pulmonary artery pressure, when the patient is in more than one position. The data collected at these different positions can be used in conjunction with each another (for example, a ratio of data sitting and supine) to determine patient health.

In some embodiments of the airway device/controller, the user is prompted to breathe in one or more specific ways to obtain the pressure signal. The user may be asked to breathe (exhale, inhale or both or neither or MVM) into the device while simultaneously the controller controls a display which displays feedback on the pressure, the time, or other parameter, of the user's breathing. For example, the user may be asked to exhale at a steady pressure, within a pressure range, for a certain duration of time, and the display may show the user feedback on that pressure, and time, such as lights, a graphic display, or alternatively, the controller may provide audible feedback.

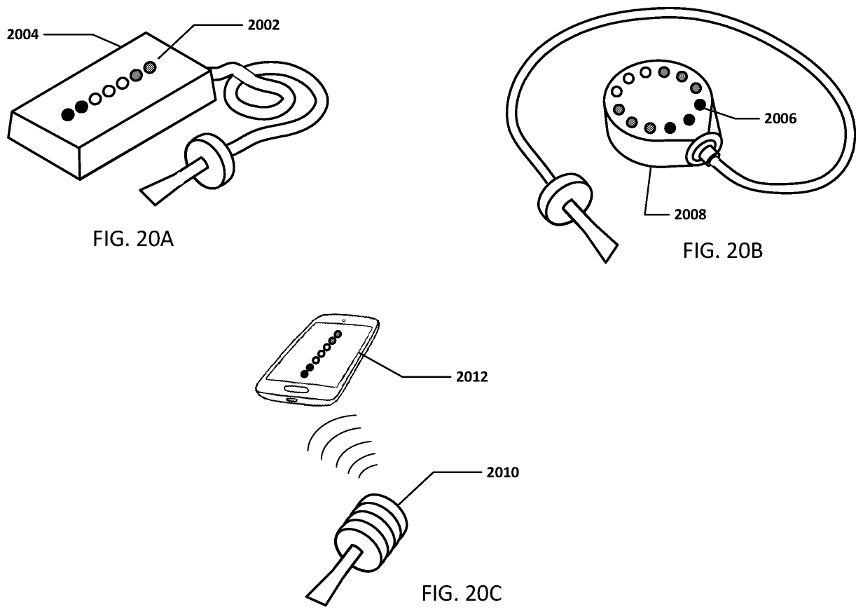

FIGS. 20A-C shows some alternative graphical displays which can guide the user to breathe at a constant pressure within a pressure range. FIG. 20A shows controller 2004 with indicator lights 2002. In this example, there are three ranges of lights with different colors, brightness, shape, etc. The lights indicate when the exhale pressure is too high, too low, or in range. In this example, the middle 3 lights show that the exhale pressure is in the optimal range. The user exhales into the mouthpiece and tries to hold his/her exhale pressure within the middle lights. Alternatives to lights may be used, including a graphical image, sounds, a dial, etc.

The graphical (or audible) display may also show the user when he/she is at the lower or higher end of the goal range (such as the pressure range), so that the user can make small adjustments to keep within range.

In some embodiments the goal exhale pressure is about 7 to about 8 mmHg. In some embodiments the goal exhale pressure is from about 9 to about 11 mmHg. In some embodiments the goal exhale pressure is from about 5 to about 7 mmHg. In some embodiments the goal exhale pressure is from about 5 to about 20 mmHg. In some embodiments the goal exhale pressure is from about 5 to about 15 mmHg. In some embodiments the goal exhale pressure is from about 10 to about 20 mmHg. In some embodiments the goal exhale pressure is from about 5 to about 10 mmHg. In some embodiments the goal exhale pressure is from about 7.5 to about 12.5 mmHg.

In some embodiments the goal exhale time is about 5 seconds. In some embodiments the goal exhale time is about 4-6 seconds. In some embodiments the goal exhale time is about 3-7 seconds. In some embodiments the goal exhale time is about 2-5 seconds. In some embodiments, the goal exhale time is set anywhere up to 10 seconds.

FIG. 20B shows an alternative arrangement of the graphical display. In this case, lights 2006 are used, but they are in a circular or oval pattern on a more curved controller 2008.

FIG. 20C shows an alternative arrangement of the airway device/controller. In this example, mouthpiece 2010 may include sensors, filters etc., and a wireless transmitter, such as a Bluetooth transmitter. The mouthpiece may contain components of the controller as well. At least some components of the controller are included in portable device 2012. The portable device may be a mobile phone, table, computer etc., and includes a wireless receiver and a display. In this embodiment, pressure sensor information is transmitted wirelessly to the controller device which displays or communicates feedback to the user. The feedback may include breathing pressure feedback, time, as well as any other feedback. The connection between the mouthpiece and the controller may be wired as well. Different components of the controller may be distributed between the mouthpiece and the controller.

Some components of the controller may also exist remotely, for example on an internet connected server, which is in communication with the local controller.

FIGS. 21A-B show embodiments of the airway device/controller which include a “sensor hand-piece”. A sensor hand-piece includes sensors and/or electrodes on the hand-piece itself for sensing various physiological parameters. The sensors/electrodes may be on the surface of the sensor hand-piece where they are easily in contact with one or more fingers and/or hands of the user. The sensor hand-piece may include sensors such as ECG sensors/electrodes, and/or pulse oximeter/photoplethysmograph sensors/electrodes on its surface. In this embodiment the sensor hand-piece is an ergonomic case which the user holds so that his/her fingers are in contact with various and appropriate sensors which are on the surface of the case. In FIG. 21A sensor hand-piece 2102 includes ECG sensors/electrodes 2104 and pulse oximeter/photoplethysmograph sensor/electrode 2106. The user may hold the sensor hand-piece with both hands so that 2 fingers/thumbs are touching the ECG sensors and another finger/thumb is touching the pulse oximeter sensor. Alternatively, the different types of sensors may be combined so that fewer fingers are required.

FIG. 21B shows a sensor hand-piece connected to a controller. This connection may be wireless or wired. Alternatively, the sensor hand-piece may be combined with the controller—the controller/sensor hand-piece may include a graphical display as well as sensors.

In some embodiments the airway device/controller is incorporated into a CPAP (Continuous Positive Airway Pressure) device. In these embodiments the controller may control the positive pressure delivered by the CPAP device based on the controller's analysis of the cardiogenic oscillation pressure curve.

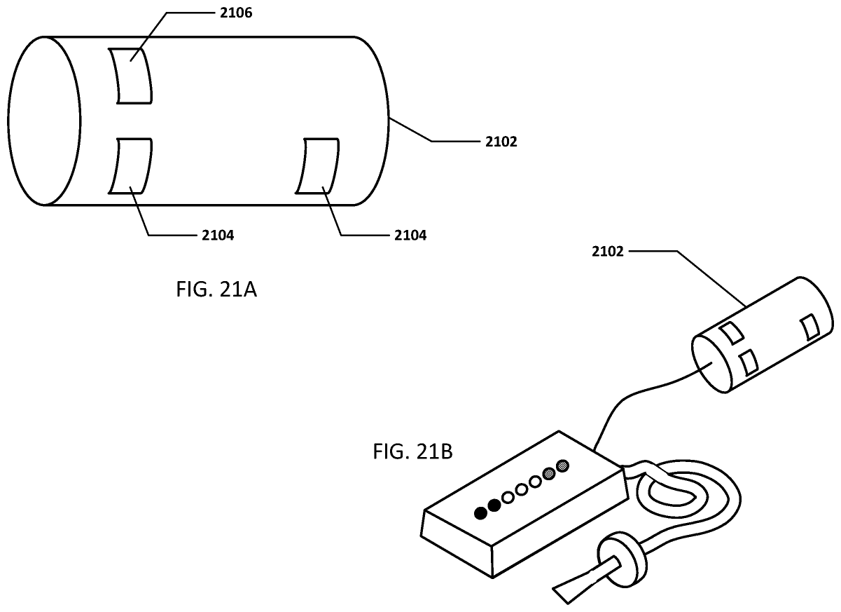

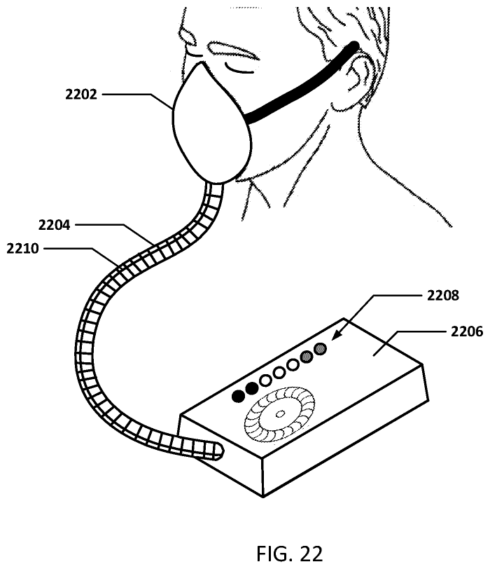

FIG. 22 shows an embodiment of the airway device/controller which has been incorporated into a CPAP device. The various sensors may exist in mask 2202, ventilation tubing 2204 or controller 2206. A handheld unit, such as the one shown in FIGS. 21A/B may also be present for use when the user is awake, and may or may not include sensors such as ECG and photoplethysmograph sensors. Indicator graphics 2208 may also be present for setup, or for when the user is awake. Sensors in the mask may communicate with the controller via wire 2210 embedded in the ventilation tubing or the communication may be wireless. No communication may be necessary if the sensors are located in the controller rather than the mask. Cardiogenic oscillations and other parameters (such as ECG, pulse, etc.) may be sensed via the CPAP system via sensors in the mask, the controller, or elsewhere, such as in a sensor hand-piece. The CPAP device may be run in a test or setup mode, to obtain readings when the user is awake, and also in a sleeping mode, to obtain readings while the user is sleeping. For example, the user may be prompted to breathe at a certain pressure for a certain time frame while awake to set a baseline. Readings may then be collected while the user is sleeping and adjustments to the CPAP airway pressure may be automatically adjusted based on changes to the readings. In addition, or alternatively, an alert may sound to wake the user when certain changes in readings are detected.

In embodiments of the airway device which are combined with a CPAP device, or other positive pressure device, heart health may not only be diagnosed, but treated. Because the airway device measures the exact timing of heartbeats (via ECG, photoplethysmograph, rough breath pressure, cardiogenic oscillations, etc.), positive pressure can be applied through the airway to the lungs in synchrony with the heartbeat. Pulmonary pressure can be increased after ventricular contraction, and decreased before the next heart contraction, to offload the work of the heart.

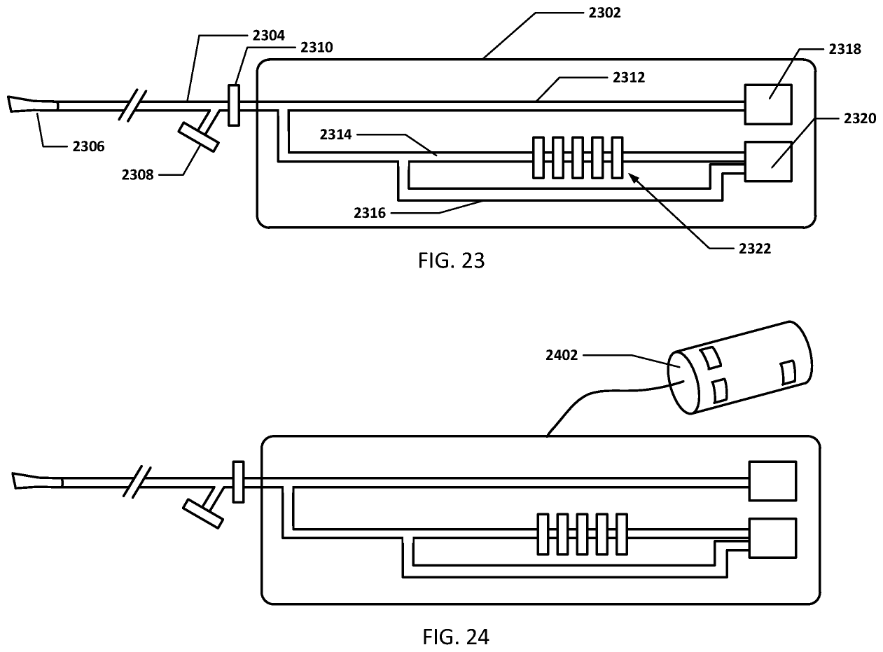

FIG. 23 shows an embodiment of the airway device/controller which may be standalone, or incorporated into a CPAP or other device, or used with additional controller components. Case 2302 houses the mechanisms of the controller. Airway tubing 2304 is in fluid communication between the controller and mouthpiece 2306. In this embodiment, all of the sensors and the controller are within casing 2302, but other embodiments may exist with sensor and controller functions elsewhere, for example as shown in FIG. 24 where sensor hand-piece 2402, including ECG and pulse oximetry sensors, is shown, and/or in embodiments where some or all of the controller computational functions are performed on a remote server.

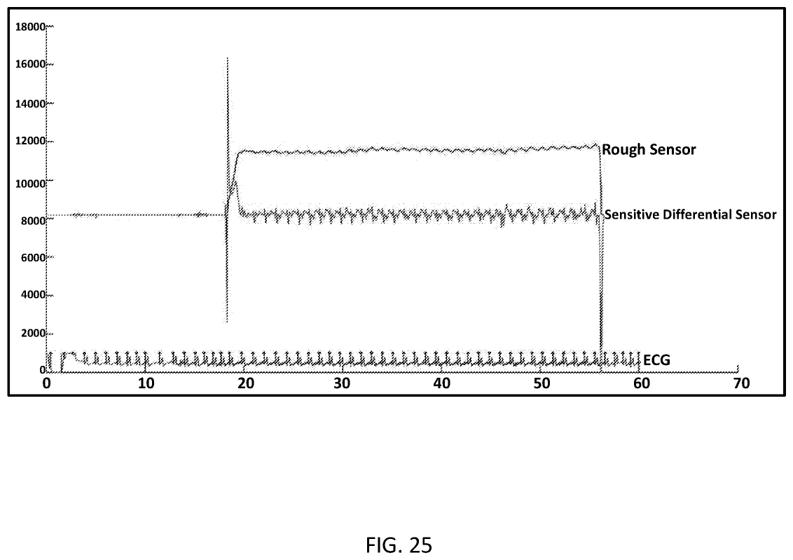

Mechanical filters 2308 and 2310 help restrict and control the flow of air through the airway tubing. Inside the case, rough pressure sensor tubing 2312 is in fluid communication with rough pressure sensor 2318 and airway tubing 2304. Fine differential pressure sensor 2320 is in fluid communication with tubings 2314 and 2316 which are in fluid communication with rough pressure sensor tubing 2312. Inline flow restrictor 2322 is incorporated into tubing 2314 to restrict flow through one tubing in fluid communication with fine differential pressure sensor 2320. The other tubing, tubing 2316, either does not have a flow restrictor, or has a flow restrictor with a different restriction level than flow restrictor 2322. Flow restrictor 2322 may comprise one or more mechanical filters. The signal from rough pressure sensor 2318 may be subtracted from the signal from fine differential pressure sensor 2320 to remove artifacts from breathing, moving, coughing, etc. See FIG. 25. Alternatively, the pressure signal from the rough sensor may be used to determine that the user is exhaling at a constant and targeted pressure. For example, the rough pressure sensor signal may drive the graphic display, such as the lights to show the user when he/she is in the target pressure range. In this situation, the cardiogenic oscillation signal would be extracted from the fine differential pressure sensor.

In its simplest form, the airway device may include one pressure sensor, and no flow restrictors or mechanical filters (except possibly a filter to prevent contamination of the breathed air) in addition to the controller functions to process the pressure signals in the breath. This embodiment, and/or any embodiment disclosed herein, may be used in a handheld format, in conjunction with a CPAP or ventilator or other device, or as a standalone device. The controller functions may be integrated into the airway device or may be separate, for example in a mobile device or other computer-type device. The single pressure sensor may be a non-differential pressure sensor.

Fine pressure sensor sensitivity may be around +/−2 mmHg. Rough pressure sensor sensitivity may be around +/−10 mmHg. Alternatively, fine pressure sensor sensitivity may be around +/−0.5 mmHg-+/−3 mmHg. Rough pressure sensor sensitivity may be around +/−5 mmHg-+/−15 mmHg. Fine pressure sensor or rough pressure sensor may be differential pressure sensors.

As mentioned elsewhere herein, pulse transit time may be determined by evaluating the time between the ECG signal (for example, the R peak) and the heartbeat as measured by pulse oximetry/photoplethysmograph. Pulse transit time may also be determined by determining the time between ECG signal and a peak in the cardiogenic oscillation curve. This may provide different information including heart valve opening pressure and/or opening times.

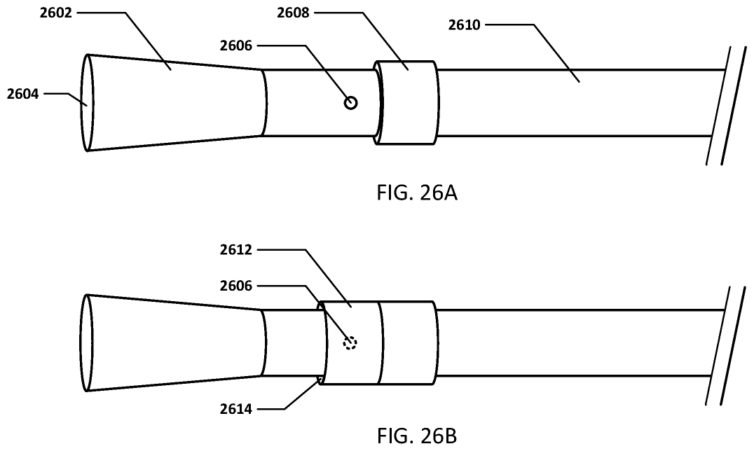

FIGS. 26A and 26B show 2 embodiments of the mouthpiece area of the airway device. Mouthpiece 2602 with mouthpiece opening 2604 is in communication with filter segment 2608 which is in communication with airway tubing 2610 so that a lumen runs from mouthpiece opening 2604 to the controller (not shown). Filter segment 2608 contains a hydrophobic filter membrane with a pore size ranging from about 2 micron to about 4 micron. Alternatively, the pore size of the filter ranges from about 1 micron to about 5 micron. Preferably, the filter pore size prevents water vapor from passing through the filter.

This embodiment includes resistance control orifice 2606 which controls the resistance felt by the user while blowing through the mouthpiece. In some embodiments, the user is asked to exhale at a relative constant exhale pressure for about 1 to 5 or about 5 to 10 seconds. The resistance control orifice can control the exhale resistance. A smaller sized resistance control orifice will equate to a higher exhale resistance. A larger sized resistance control orifice, or multiple resistance orifices, will result in a lower exhale resistance. Preferably, the resistance control orifice is approximately circular in shape and ranges from about 0.3 mm to about 0.5 mm in diameter or longest dimension. Alternatively, the longest dimension of the resistance control orifice is from about 0.1 mm to about 1.0 mm. Alternatively, the longest dimension of the resistance control orifice is from about 0.5 mm to about 1.0 mm.

Filter segment 2608 may adapt to mouthpiece 2602 and/or airway tubing 2610 by a common luer type adapter or any other suitable adapter such as luer-lock, screw on, snap on, glue on adapter etc.

FIG. 26B shows an embodiment similar to that shown in FIG. 26A with the addition of orifice sheath 2612. The orifice sheath protects the resistance control orifice from blockage by the users finger or otherwise. The orifice sheath includes opening 2614 which allows air to freely flow out of (or into, during inhalation) the resistance control orifice even if the user has his/her fingers on the orifice/orifice sheath area. The orifice sheath is preferably made from a rigid material such as a polymer, and may be integral with mouthpiece 2602 and/or filter segment 2608.

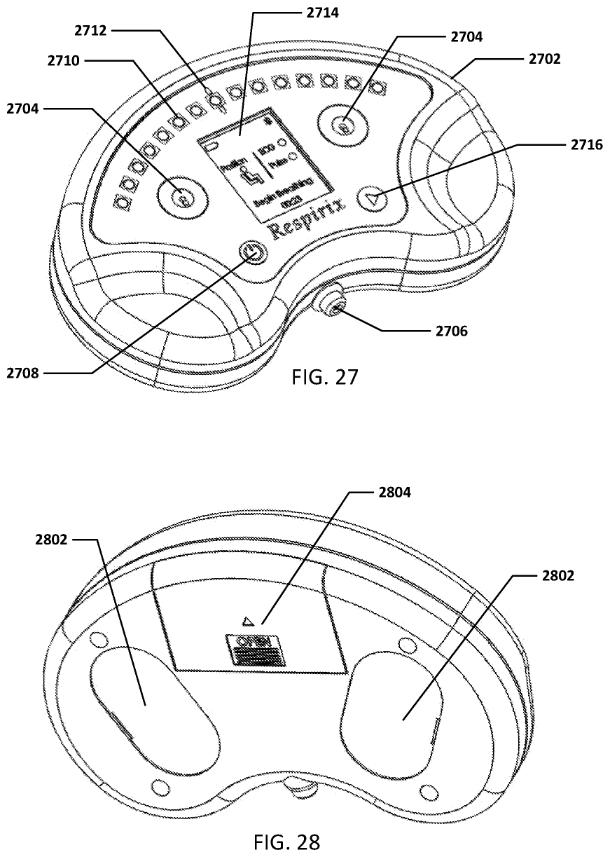

FIGS. 27 and 28 show the top view and bottom view, respectively, of an embodiment of a sensor hand piece of the airway device which includes at least some of the controller functions. The sensor hand piece includes casing 2702. On the casing top are one or more heart pulse sensors/electrodes (photoplethysmograph/pulse oximetry sensors) 2704, airway tubing connector 2706, power button 2708, breath pressure indicator or indicators 2710, breath pressure goal indicator or indicators 2712, display 2714, and mode button 2716.

FIG. 28 shows the bottom view of an embodiment of a sensor hand piece including ECG electrode or electrodes 2802 and battery cover 2804. Note that any of the sensors/electrodes may be anywhere on the case.

When holding the sensor hand piece, a user will preferably have one or both thumbs in contact with pulse sensor(s) 2704, and one or more fingers in contact with ECG electrode(s) 2802. Preferably, before the user places his/her fingers/thumbs on the sensors, he/she will place the mouthpiece in his/her mouth. However, if only one hand is necessary for the sensor contacts, the mouthpiece may be placed in the user's mouth at any time before testing begins.

During data collection, the user is guided through various steps either by display 2714, or audibly or both. Alternatively the display may be on the user's mobile phone/computer with other functions of the sensor hand piece in communication with the mobile phone/computer either by a wired connection, a wireless connection, or direct connection via a port in the mobile phone/computer. Indicator(s) 2710 may be lights, a visual bar, audible sounds, tactile feedback (such as vibration) etc. in this figure, the indicators are lights which indicate the exhale pressure as well as the consistency of the exhale pressure, and may also indicate when the exhale pressure has been consistently in the proper pressure range for the required time. Preferably, the user is guided by the indicators to hold a steady pressure. In addition or alternatively, the user is guided by the indicators to hold a particular target pressure. A target pressure may be preset or may be set depending on the individual user and the user's comfortable exhale pressure range for holding a steady exhale pressure. Pressure goal indicator 2712 may be fixed in place or may be movable to suit individual users. The software may also include the ability to adjust indicators 2710 so that goal indicator 2712 stays in the same location, but refers to different exhale pressures for different individuals. For example, as part of a set up procedure, the user may be asked to exhale at a comfortable pressure for x seconds. A button on the device may be pressed to set that pressure as the “goal” pressure for the particular individual.

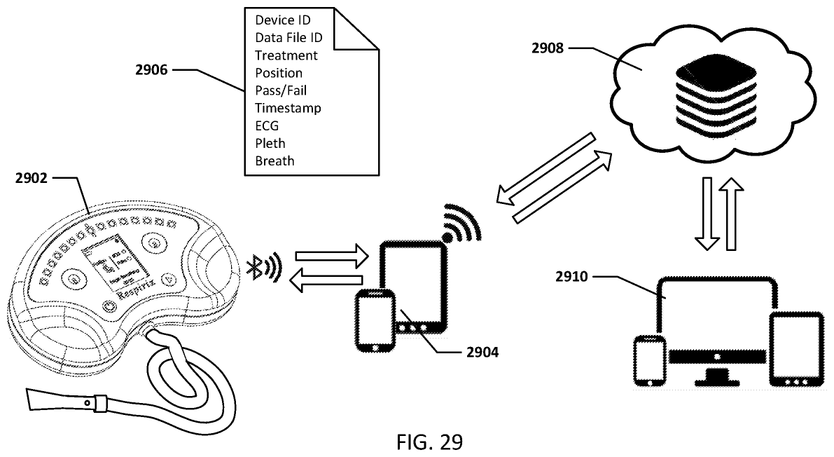

FIG. 29 shows how an embodiment of the airway device controller and the system communicates data. In this embodiment, the controller functions are distributed among sensor hand piece 2902, mobile phone/device/computer 2904, cloud/internet/intranet server 2908 and dashboard computer 2910. In general, data is collected via sensor hand piece 2902, communicated locally with mobile device 2904 (via Bluetooth, wifi, or other wired or wireless connection) which communicates remotely with server 2908 (via wifi, mobile network, wired or wireless communication etc.) which can then communicate with any device, such as a desktop computer, laptop computer, mobile device, tablet, client, etc. (via wifi, mobile network, wired or wireless communication etc.).

Data 2906 transferred from the sensor hand piece includes Device ID (the unique identifier of the hand piece, which may be in the form of a MAC address or other ID), Data File ID (the unique identifier of the data file within the device), Treatment data, Position data (sitting, lying down, etc.), Pass/Fail data (related to whether sensor data collected is adequate for analysis, may also be adequacy level data), Timestamp(s), ECG data, pulse oximeter/photoplethysmograph data, Breath data, and other relevant data.

Sensor hand piece 2902 may incorporate any of the embodiments disclosed herein. Server 2908 may be remote or local, and may be on the internet, intranet or local network. Dashboard computer 2910 may have different access rights. For example, the patient/airway device user may have one access level, where a physician or an insurance company or a clinical trial administer or another type of administrator may have another access level. Data may be displayed in various ways depending on the user. For example, a clinical trial administrator may see data aggregated from more than one user, but may not see the identity of the users. The patient may see only his/her data and may see trends, alerts, suggestions etc. The patient's physician may see the data of several patients, each identified, with alerts, data trends, etc. The dashboard computer, or server, may be integrated with an electronic health record. Data from the airway device may be integrated with a patient's personal electronic health record. Data from the airway device may be used to make diagnoses either with or without data from other devices/sources. Server 2908 may include algorithms which incorporate and analyze data from the airway device and optionally other data collection devices to predict outcomes.

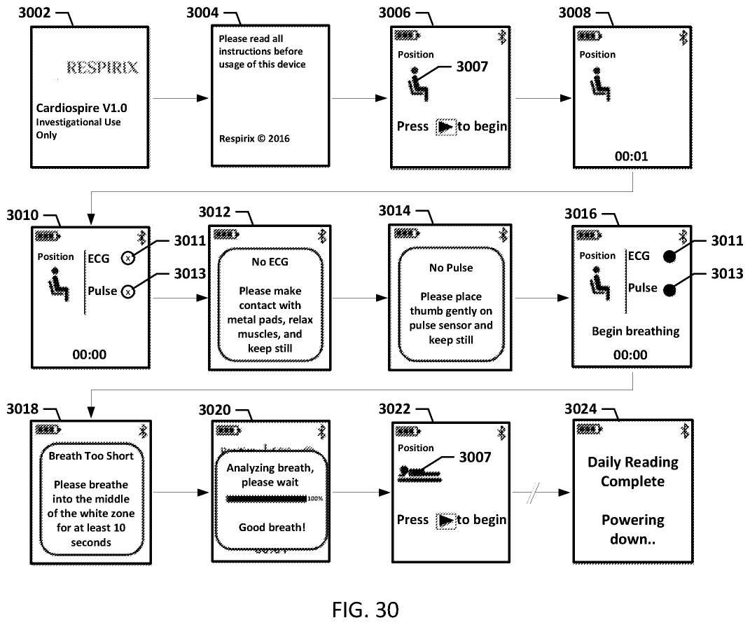

FIG. 30 shows examples of screens that may be shown on display 2714 shown in FIG. 27 or on a mobile device etc. Screen 3002 shows a sample welcome screen. Screen 3004 shows a sample information screen. Screen 3006 shows a sample start screen, which includes positional indicator 3007 indicating in which position the user will start. For example here, the user will start in the sitting position. Other positions may include supine, supine with legs raised, standing, etc. A Bluetooth indicator shows that the user is connected to his/her mobile device via Bluetooth and the user is encouraged to push the mode button to begin the testing. The user may see more information including how to hold the hand piece and where to place his/her finger(s) and thumb(s). Screen 3008 shows that the testing has now begun. First the system will test to confirm that adequate contact with the sensors is sensed, by determining whether the ECG and pulse signals are adequate. “Adequate” or “good” may include measuring the signals over time to determine magnitude, consistency, shape, trends, or other attributes of the signals. Screen 3010 shows ECG signal adequacy indicator 3011 and pulse signal adequacy indicator 3013 show an inadequate signal until an adequate signal is obtained. The indicators may show a red indicator, an open indicator, an X, or a sound or tactile indicator, such as a vibration which tells the user when the signal(s) is inadequate and/or adequate. Screen 3012 is a screen showing the user that the ECG signal is inadequate. This type of screen may pop up after multiple attempts at an adequate signal or when a signal has been inadequate for a certain period of time. Additional instruction may be provided to help the user obtain an adequate signal. Screen 3014 shows a similar screen when an adequate pulse signal has not been obtained.

Screen 3016 shows a screen that indicates that adequate ECG and pulse signals are being obtained. The user is then instructed to begin breathing. Breathing may mean natural breathing or a prolonged steady exhale or inhale. Preferably, the user is asked to produce a steady prolonged exhale. Again, the system evaluates the signal to determine if the breath signal is adequate or inadequate. The adequacy of a breath signal may include length of signal, consistency of signal, magnitude of signal, shape of signal (such as the pressure signal), existence of regular peaks, etc. Screen 3018 shows a sample screen which may be displayed if the breath signal is determined to be inadequate. The screen may provide additional information such as length of breath, steadiness of breath, magnitude of breath etc. For example, here the user is asked to breath for at least 10 seconds. The user is also asked to breath in the middle of the “white zone”, meaning to breathe so that indicators 2710 shown in FIG. 27 are in the goal range. Keeping the indicator in the goal range will control both the amplitude of the breath signal as well as the steadiness of the breath signal. All of these prompts may be provided to the user or a different prompt may be provided depending on whether length, amplitude, or steadiness of the signal is lacking.

Screen 3020 shows the progress of the test when breath, ECG and pulse signals are all adequate. The system may analyze the results in real time to determine whether the total signal is adequate, meaning it can be analyzed properly. If the signal cannot be analyzed property, the user may be prompted to repeat the test.

Screen 3022 shows the beginning of the next test, which differs from the previous test by position. This screen, for example, shows that the user should now lie down. Once the user is lying down and presses the mode button, screens similar to those shown in screens 3008 through 3020 will be shown. Other factors in addition to position may be changed for different tests. For example, the user may be asked to exercise between tests, or breathe differently or for a different length of time. Screen 3024 is a sample end screen when all testing for the session is complete.

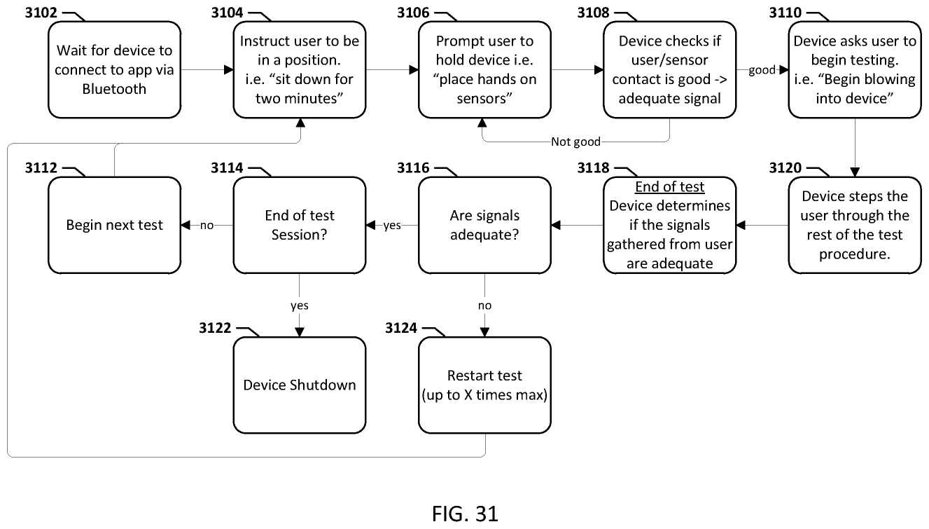

FIG. 31 outlines a method of use of an embodiment of the airway device/controller device. These are steps that the controller performs in one embodiment. Step 3102 represents the controller in standby, waiting for a user to initiate the testing by connecting the hand piece to a mobile device via Bluetooth or other technology. Once the user has initiated the test, and connected via Bluetooth, the controller prompts the user to take a certain position, such as sitting, standing or lying down, as represented in step 3104. Step 3106 represents the controller prompting the user to put his/her finger(s) and thumb(s) on the appropriate sensors. More specific instructions may be given as well. Step 3108 represents the controller checking to see if the signals are adequate. If the signals are inadequate, the controller prompts the user in a way to increase the adequacy of the signal(s). If the signals are adequate, the controller moves onto step 3110 where the user is prompted to begin testing by blowing into the mouthpiece of the device. Step 3120 represents the controller gathering data from the hand piece sensors (i.e. ECG and pulse) as well as the breath sensor(s) (i.e. pressure and/or flow). Step 3118 represents the end of the test sequence. At this point, the controller determines whether the data collected during the test sequence are adequate—represented by step 3116. If the data are adequate, then the controller ends the current test session represented by step 3114. In step 3112, the controller prompts the user as to, or communicates to the user, whether there are any more tests that need to be performed, for example, in a different position. If there are more tests to be performed, the controller returns to step 3104. If there are no more tests to be performed, the controller shuts down the device as shown in step 3122. If the collected data are not adequate, then the controller restarts the test session as is represented by step 3124, by returning to an earlier step, such as step 3104, 3106 etc.

Some embodiments of the airway device include capabilities to communicate with caregivers, such as physicians, nurses, family, neighbors, etc. This communication may happen wirelessly via a wi-fi connection or via a cell connection. Alternatively, the communication may happen in a wired configuration. This communication may be with a network or directly peer-to-peer. The network may be the internet, intranet or other network. These communications may help caregivers monitor a user's health status. For example, data may be communicated which relates to congestive heart failure, PAC, pulmonary issues (such as COPD, emphysema, asthma, lung capacity, lung sounds, asthma, shortness of breath, etc.), other cardiac issues (such as atrial fibrillation, valve regurgitation or prolapse, plaque buildup, heart murmurs, heart sounds, etc.), diabetes, stroke, nutrition, medication adherence, routine adherence, physical strength, physical dexterity, hydration, temperature, blood pressure, heart rate, respiratory rate, tidal volume, ECG, breathing sounds, saliva chemistry, breath chemistry, steadiness/tremors, hearing, etc. These conditions may be monitored over time and changes in patterns may indicate a problem and may be communicated to one or more caregivers. Alternatively, certain thresholds may be predefined or “learned” from the data which trigger an alert to a caregiver. Effectiveness of various treatments may also be monitored over time. In addition, the airway device may be able to differentiate between conditions that share similar symptoms. For example, the device may be able to differentiate between COPD, or other pulmonary issues, and CHF.

In situations where data is transmitted via a network, privacy is of utmost concern. The data may be encrypted, anonymized etc. to adhere to HIPAA (The Health Insurance Portability and Accountability Act of 1996) standards. In addition, the identity of the user may be confirmed based on data consistency (data for a session is similar enough to the data of past sessions), fingerprint ID (may be gathered from the pads on the handheld portion of the device or elsewhere, such as on a mobile phone screen), DNA ID (may be gathered from saliva or elsewhere), survey questions etc.

More specifically, pulmonary diseases, such as COPD, emphysema, asthma etc, may be monitored by a spirometer, or other way of measuring breath flow. In some embodiments, the device is able to differentiate between COPD and cardiac conditions, even when symptoms are similar, by analyzing data from both a spirometer and COS data from a pressure sensor. For example, breath flow may be measured by ultrasonic, Doppler, mechanically (as in a pinwheel type device) etc. A microphone may be incorporated into the airway device to detect lung sounds such as crackling or wheezing, which may be an indicator of fluid in the lungs or other problems with the lungs. Adherence and/or effectiveness of medications may be monitored by looking at this type of data over time, to see if symptoms/conditions worsen or get better. Also, chemical markers in the breath and saliva can be monitored to confirm use of certain medications/treatments.

In addition to monitoring heart failure conditions (described extensively herein), other cardiovascular conditions such as AFIB (atrial fibrillation), valve issues (regurgitation, irregularities, etc.), plaque buildup may also be monitored. For example, a microphone may be incorporated into the airway device to detect heart sounds such as those associated with valve regurgitation, irregularities, and/or atrial fibrillation. One or more ECG sensors may be incorporated into the handheld portion of the airway device or elsewhere on the device to monitor the ECG. In addition, PAC (described elsewhere herein) is also useful in monitoring heart conditions other than heart failure. For example, PAC may be used to monitor the buildup of plaque, or other issues within the cardiovascular arteries.

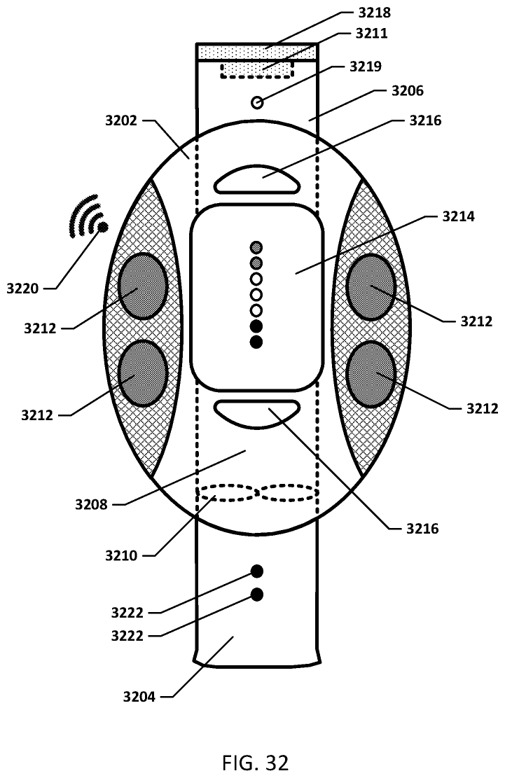

FIG. 32 shows an embodiment of the airway device which incorporates a spirometer, or flow meter. Sensor hand-piece 3202 includes mouthpiece 3204, endpiece 3206, inner lumen 3208, which connects the mouthpiece and the endpiece, pinwheel 3210, pressure transducer 3211, electrodes or sensors 3212 (which may be on top and/or bottom of the sensor hand-piece), display 3214, buttons 3216, cap 3218, resistance control orifice 3219, and transmission capability, 3220. This embodiment may also include other sensors 3222, such as a temperature sensor, microphone, analyte sensor, etc. either inside the mouthpiece/inner lumen/endpiece, or on the outside of the mouthpiece, in contact with the mouth. A temperature sensor may be inside the mouthpiece, or on the outside of the mouthpiece. A microphone may be inside the mouthpiece or inner lumen. An analyte sensor may be on the outside of the mouthpiece.

The airway device shown in FIG. 32 can be used to diagnose and/or monitor pulmonary diseases, such as COPD, emphysema, asthma, in addition to monitoring cardiovascular health. The device is used with cap 3218 removed to assess pulmonary function. In this mode, the user is prompted by the controller to breathe through mouthpiece 3204, either regularly, or in a forced exhale, so that air flows through mouthpiece 3204, past sensors 3222 which may assess breath temperature, sounds, etc., past pinwheel 3210, which senses air flow, and through inner lumen 3208 and exits the device via endpiece 3206, which is open in this mode.

The device is used with cap 3218 in place to assess cardiovascular health. In this mode, the user is prompted by the controller to contact sensors 3212 in a particular way (sensor contact may also be requested by the controller in pulmonary function mode). The user is also prompted by the controller to exhale into the mouthpiece with the throat open, for example via a MVM. Indicators on display 3214, or audible or tactile indicators may help the user perform the MVM at the required pressure for the required period of time, and in the required position(s). Buttons 3216 may serve other functions, such as on/off, display settings, navigation etc. Data may be transmitted from the device wirelessly via Bluetooth, wifi, cellular network etc. Data may also be transferred via a wired connection, such as USB. Pressure transducer 3211 may be incorporated into cap 3218 or may be incorporated into the sensor hand-piece body. Display 3214 may be on the sensor hand-piece body, or may be on a mobile device, such as a mobile phone or tablet, or the display may be incorporated into both devices.

As mentioned elsewhere herein, cardiogenic oscillations are detectable during a “modified Valsalva maneuver” or MVM, where the user has his/her throat open (i.e., leaving the glottis and/or epiglottis open) during exhalation against pressure. This is referred to as a “modified Valsalva maneuver” or MVM. The patient/user may be prompted to exhale within a specific pressure range and for a specific time period. The user may be prompted by the controller, or instructed, to perform the MVM within the proper parameters (open throat, pressure, and time).

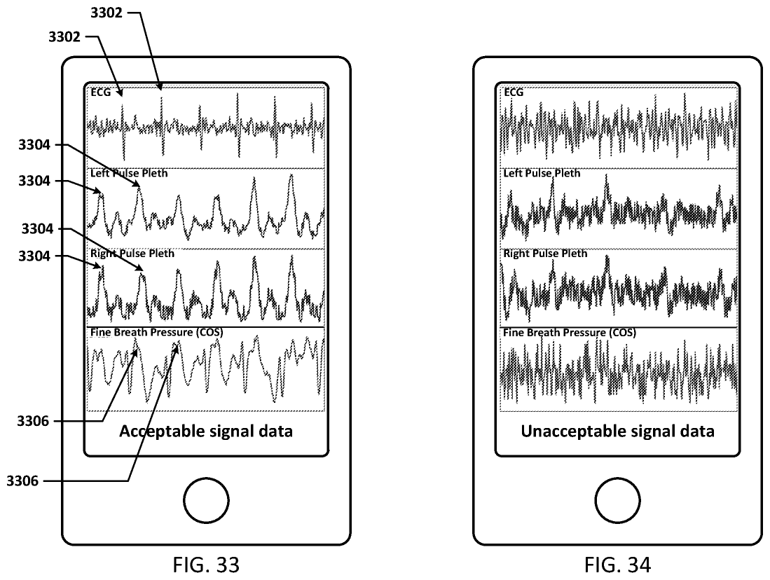

To ensure that the user is performing the MVM properly, the user may be instructed to perform use a training app. The training app may be part of the controller function (i.e., an app on a mobile phone, which is in communication with a sensing hand-piece). The training app may prompt the user to place the mouthpiece in his mouth, and place his fingers/thumbs on the sensors on the sensor hand-piece. The training app may then ask the user to exhale into the mouthpiece while holding the sensor hand-piece. The user will be asked to exhale with his throat open, at a steady pressure indicated on the display, for a set time period. For example, the user may be asked to exhale at a steady pressure, for 5 seconds, so that a light indicator on the display remains within an indicated range. The app may then display the results of the test. Examples of displayed data are shown in FIGS. 33 and 34. FIG. 33 shows examples of acceptable ECG data, left photoplethysmograph data, right photoplethysmograph data, and cardiogenic oscillation (breath fine pressure) data. FIG. 34 shows examples of unacceptable ECG data, left photoplethysmograph data, right photoplethysmograph data, and cardiogenic oscillation (breath fine pressure) data. In some embodiments, the user (or a physician, assistant, technician or nurse) will be prompted to visually assess the data curves to determine whether they are acceptable or unacceptable. For example, an acceptable ECG curve will have noticeable and periodic R-wave spikes 3302 that represent the user's heartbeat. An acceptable photoplethysmograph curve will have noticeable and periodic spikes 3304 that represent the user's heartbeat. An acceptable COS curve will have noticeable and periodic peaks 3306.

In some embodiments, the signal curve data for the various sensors is analyzed for shape in a similar manner by the controller. In these embodiments, the data may not be displayed on the display. The controller (or user) may only need one acceptable gating signal (ECG or either photoplethysmograph signal) and an acceptable COS signal to prompt the user to continue to the actual data collection. For example, if the left photoplethysmograph signal and the COS signal are acceptable, the left photoplethysmograph signal may be used to gate the COS signal.

If the ECG signal is unacceptable, the user may be prompted by the controller to contact the ECG sensor differently, for example with more pressure, less pressure, or with more sensor coverage. If the photoplethysmograph signal is unacceptable, the user may be prompted by the controller to contact the ECG sensor differently, for example with more, less pressure or with more sensor coverage. If the COS signal is unacceptable, the user may be prompted by the controller to breathe differently, for example, to hold breath more steady (if rough pressure sensor has determined that the exhale pressure was not within range for long enough, or if COS signal curve does not have identifiable peaks), to exhale for longer (if rough pressure sensor has determined that the exhale pressure was not within range for long enough or if not enough identifiable peaks are available), to exhale with more or less pressure, to open his throat while exhaling (if COS curve does not have identifiable peaks) etc.

Once the user has obtained acceptable data via the training app, he may move on to actual data collection for analysis and trending. The user may periodically be prompted by the controller to fill out a survey concerning his/her health. The survey may be requested once/data collection session, or at any other interval, for example once/day, once/week, once/month, etc.

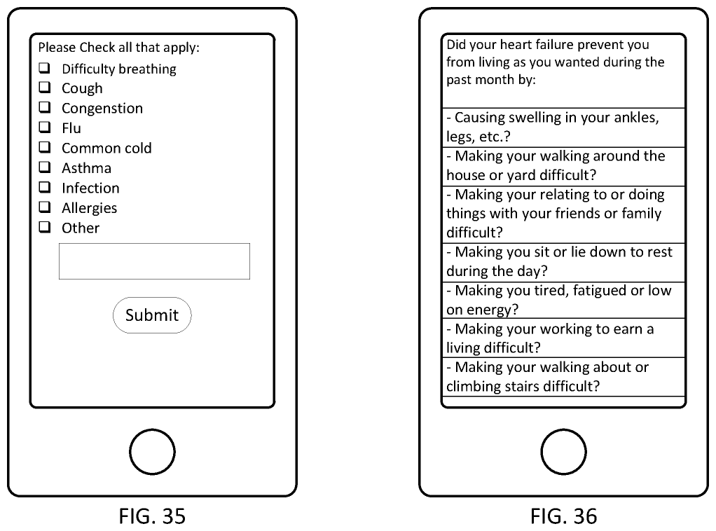

FIGS. 35 and 36 show examples of survey screens displayed to the user by the controller. One example of a survey which may be used is the Minnesota Living With Heart Failure Questionnaire.

Some embodiments of the airway device may include one or more accelerometers on the handheld portion of the device. Data from accelerometers may be used to sense the position of the device to ensure proper positioning (for example, is the subject lying down or sitting based on the angle), or the existence or worsening of tremors.

Some embodiments of the airway device may include a speaker to introduce sounds into the mouthpiece/tube. Some embodiments may include a hearing test—for example, touching certain sensors on the handheld portion in response to auditory tones created by the controller.

Some embodiments of the airway device may include impedance sensors (for example on the mouthpiece, or on the handheld portion, contacting one or both hands). For example, 2 separate impedance sensors may be on the handheld portion—one for the left hand and one for the right hand, so that impedance measurements can be taken across hands. Impedance measurements can determine user hydration. Data from the impedance sensors may be used alone or in conjunction with data from other sensors. For example, impedance data may be used in conjunction with PAC data to determine user hydration. Impedance measurements may also be used to determine user body weight and/or fat composition.

Some embodiments of the airway device may be able to monitor diabetes, for example by monitoring analytes in saliva, breath or on the skin.

Some embodiments of the airway device collect data on usage patterns in addition to physiological data to determine whether the user is straying from routine. Straying from routine may be an indicator of a health issue such as stroke, dementia, etc.

Some embodiments of the airway device include one or more strain gauges on the handheld portion to determine finger pressure. These data may be used for device usage compliance (is the user positioning his/her fingers appropriately on the device) and/or for physiological data such as finger strength or steadiness. Some embodiments may include a dexterity test, where the user is asked to touch certain sensors quickly, or in a test pattern, which is indicated by sounds and/or lights or other indicators produced by the controller.

Some embodiments of the airway device are calibrated using data across multiple users of multiple disease states. Alternatively or additionally, some embodiments of the airway device may be calibrated using data learned from the subject user over time, optionally augmented with surveys or other means of obtaining health related information.

Some embodiments may use a breath pressure profile to determine systolic and/or diastolic pulmonary blood pressure or pulmonary artery pressure. Pulmonary artery pressure may be useful to diagnose and/or track certain conditions including fibrosis, hypertension etc. Pulmonary blood pressure may be determined by analyzing the breath pressure profile curve obtained using the airway device over different pressures. For example, a user may be asked to exhale at a certain pressure for a certain number of seconds, the user may then be asked to exhale at an increased pressure for a certain number of seconds and so on. In this way, the breath pressure profile can be measured over a range of exhale pressures. The pressures may be increased in a step-wise fashion, or may be ramped, or may be decreased and the related breath pressure profiles analyzed.

At a pressure correlated to the pulmonary artery pressure, the breath pressure profile curve may change, for example, the curve may flatten out, or the standard deviation of the cardiogenic oscillations may decrease. The pressure at which this flattening occurs may be indicative of either systolic or diastolic pulmonary artery pressure. Other shape aspects of the curve may also be used in the analysis, including slope, rate of change of slope, magnitude, variability, shape, value, etc. Other sensor data may also be incorporated into the analysis performed by the controller including any type of sensor data disclosed herein. Data from a ventilator, a CPAP machine or other device which may affect pulmonary blood pressure may also be incorporated into the analysis.

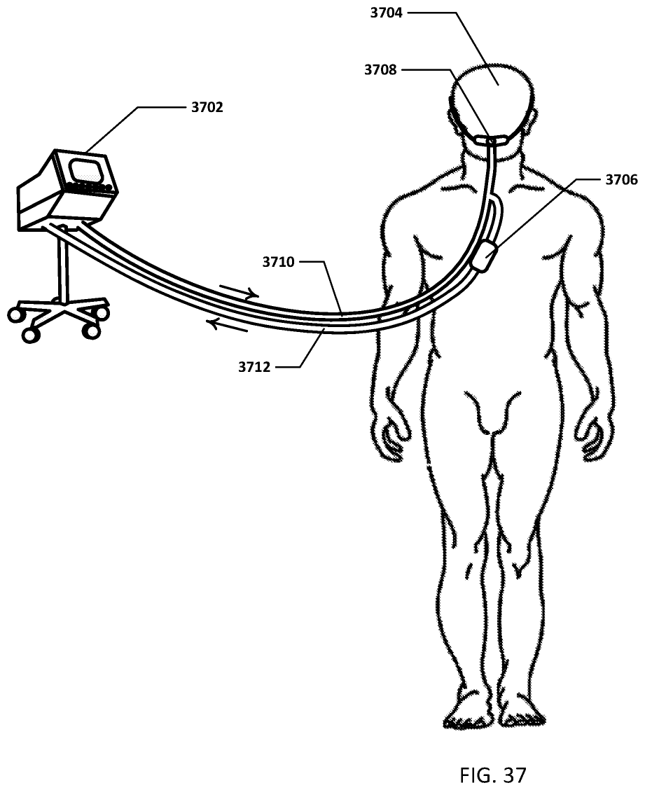

FIG. 37 shows an embodiment of the airway device/controller being used in conjunction with a ventilator, or other assisted breathing device. Ventilator 3702 is mechanically ventilating patient 3704. Airway device 3706 is shown in line with expiratory line 3712. Also shown is inspiratory line 3710 and endotracheal tube 3708. An endotracheal tube is shown here, but alternatively, a mask may be used to ventilate the patient. Although the airway device is shown in line with the expiratory line here, it may be connected to the ventilator in any manner which allows it to be in fluid communication with either the expiratory or inspiratory lumens of the expiratory and inspiratory lines, or in fluid communication with the airway of the patient. The airway device may be anywhere in the system where this is the case, including near the patient, in the patient, near the ventilator, elsewhere in either the expiratory or inspiratory lines, within the endotracheal tube, within a mask, in fluid communication with a mask/endotracheal tube, at the junction of the endotracheal tube or mask and the inspiratory/expiratory tubing, or within the ventilator. The airway device may be integrated with, or an add-on to, a ventilator system and/or mask and/or endotracheal tube.

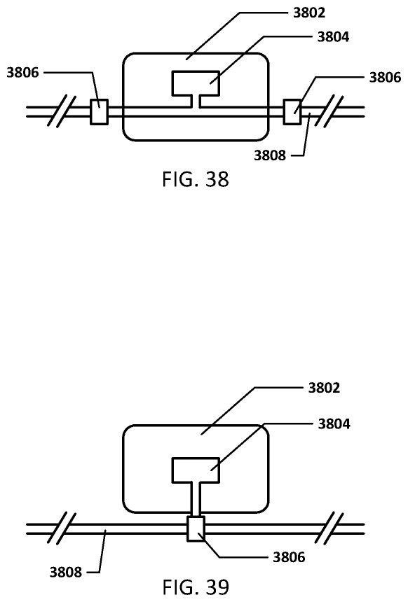

For example, the airway device may be attached to the tubing or other component of the ventilator system as shown in FIGS. 38 and 39. In FIGS. 38 and 39, airway device 3802 includes pressure sensor 3804 and may be connected to ventilator or other device components via connectors 3806. All or some of the controller functions may be incorporated into airway device 3802, and/or the airway device may communicate with a remote device, such as a smartphone or tablet, which controls all or some of the controller functions. FIG. 38 shows the airway device in line and in fluid communication with the lumen of ventilator tubing 3808. FIG. 39 shows the airway device external to, and in fluid communication with, the lumen of ventilator tubing 3808.

Although not shown in FIG. 37, an ECG, photoplethysmograph, or other device may be used to determine the heart beat cycle for the purposes of gating the airway device signal. Alternatively, the airway pressure signal of the airway device itself may be used to gate the signal. If ECG, photoplethysmograph or other signal is used, the signal acquisition sensor may be integrated with the airway device, or may be separate.

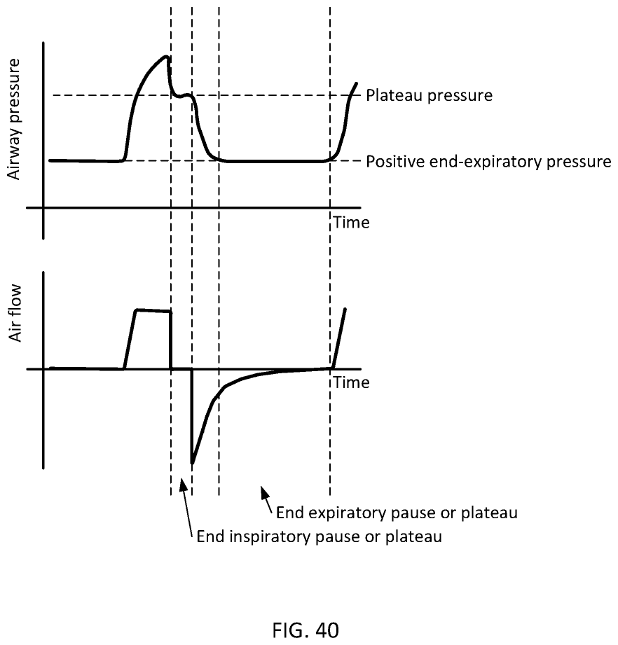

FIG. 40 shows a graph of airway pressure and air flow over time within a typical ventilator system. Cardiac oscillation pressure measurements are more easily measured during periods of ventilator pause, for example during the end inspiratory pause or the end expiratory pause. The existence of pauses, and pause lengths, may be adjusted within the ventilator settings, or may be preset. The airway pressure device may be programmed to only capture pressure data during one or more of these pauses, or may be programmed to collect pressure data during the entire airway pressure cycle, or continuously. For example, the controller of the airway pressure device may be integrated with the logic of the ventilator so that the airway pressure device knows when the pauses are, and only collects pressure measurements during the pauses. Alternatively, the airway pressure device may collect pressure data continuously and filter out the pressure data which is outside of the pauses, or filter out data which is less useful or more noisy, to obtain clean cardiogenic oscillations data.

The airway pressure device may, for example, use airway pressure data collected during a steady increase in flow or pressure caused by the ventilator. Like COS data obtained during a ventilator pause, COS data obtained during a fixed slope change in pressure can be easily separated from ventilator-caused airway pressure data.

Said another way, the airway pressure device may collect pressure data within the ventilator system throughout the breathing cycles. The logic of the controller of the airway pressure device may determine where the airway pressure pauses are, based on the quality of the pressure data. For example, during a pause, the pressure waves due to the cardiogenic oscillations may be more discernable. The COS wave amplitude may be larger compared to the variations in the airway pressure wave. COS wave patterns may be more able to be recognized during the airway pressure pause periods (or other relatively clean data collection periods). The controller of the airway pressure device can determine where the detected COS waves are long enough, and/or clean enough, to be useful in the cardiac health calculations. The COS data is likely to be more clean during the pauses, but may be obtained anywhere along the airway pressure curve. For example, the airway pressure device controller may only use COS wave data which is at least one heartbeat cycle long, or 2 heartbeat cycles long, or 3 heartbeat cycles long, Or the airway pressure device controller may only use COS wave data which is at least one second long, or at least 2 seconds long, or at least 3 seconds long, or at least 4 seconds long, or at least 5 seconds long. The COS data capture periods may be at end inspiratory, end expiratory, or elsewhere within the airway pressure cycle.

In some embodiments, the airway pressure device detects ventilator pauses without logical integration with the ventilator, so in other words, the controller of the airway pressure device determines which COS data has been collected during a ventilator pause, based on the detected airway pressure itself.

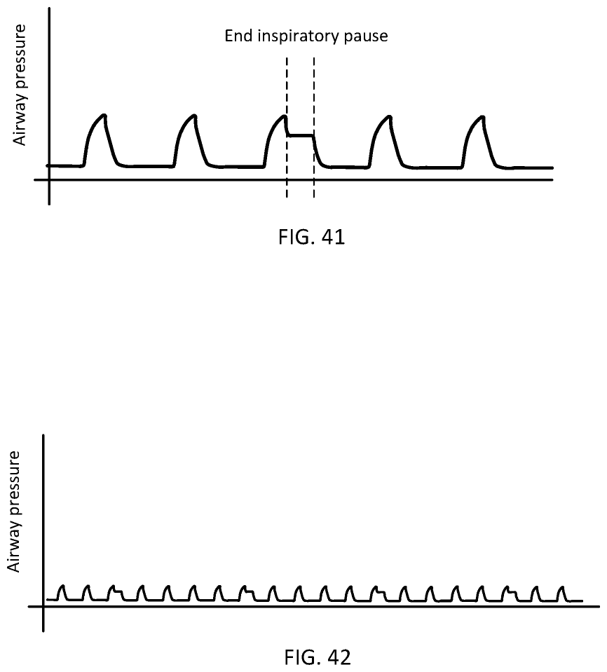

FIG. 41 shows a graph of airway pressure where an end inspiratory pause is not programmed to occur with every breath. In this situation, the end inspiratory pause may be programmed to occur every x breaths, for example every 2, 3, 4, 5, 10, 20, 60 breaths, or may be manually introduced occasionally, for example every 1, 2, 3, 4, 5, 10, 20, 60 minutes.

FIG. 42 shows several cycles of the airway pressure curve with a periodic end inspiratory pause. In this figure the pause is programmed to occur every 5 breaths.

The airway pressure device controller may use data from one or multiple pauses to determine heart health parameters. For example, the airway pressure device controller may use one heartbeat cycle of data from one airway pressure pause, and at least one more heartbeat cycle of data from a second airway pressure pause, gating the COS pressure data to the heartbeats. Alternatively, the device may use more than one heartbeat cycle of data from one airway pressure pause. Alternatively the device may use more than one heartbeat cycle of data from one airway pressure pause, and combine it with data from a different airway pressure pause. COS pressure data may alternatively be obtained elsewhere in the airway pressure curve, however it is likely the cleaner data will be available during airway pressure pauses.

In some embodiments, the airway pressure device is integrated with the ventilator logic and can instruct the ventilator to pause the airflow at least occasionally or periodically to obtain adequate COS pressure data to analyze and determine heart health parameter(s). In some embodiments, the airway pressure device controller can identify when the ventilator is pausing or has paused.

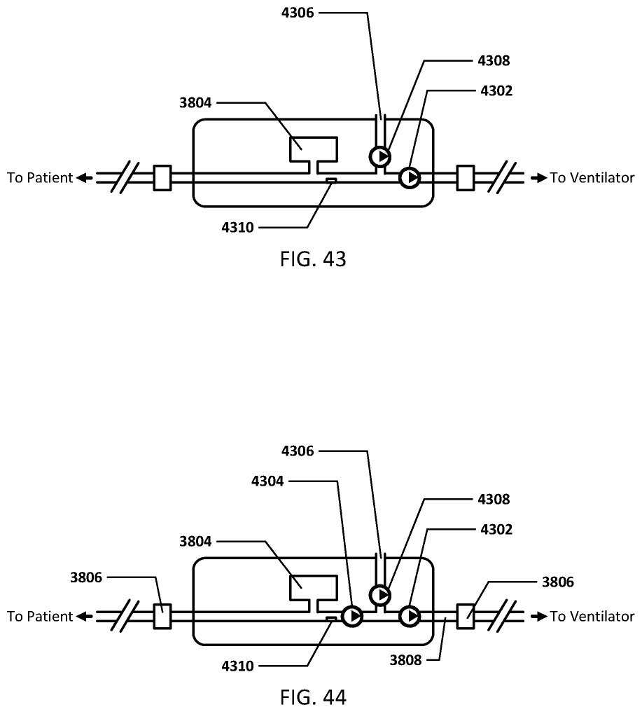

In some embodiments, the airway pressure device effectively creates ventilator pauses, in some embodiments without logical integration with the ventilator. FIG. 43 shows one of these embodiments. In this embodiment, the airway device includes valves to control the flow of ventilated air through the device. Shown in FIG. 43 are ventilator valve 4302 and exhaust valve 4306. The ventilator valve can block or allow air/gas flow to or from the ventilator via the ventilator tubing. The exhaust valve can block or allow air/gas flow through exhaust lumen 4306 which connects the ventilator tubing with atmospheric air. Also shown is optional pressure or flow sensor 4310 which can sense air/gas pressure or flow through the system.

To be clear, “without logical integration” means that the controller of the airway device is not controlling the functions of the controller of the ventilator or other assisted breathing device. In other words, the software of the assisted breathing device is not affected by the airway device in embodiments where the airway device does not logically integrate with the assisted breathing device. In these embodiments, the airway device may integrate physically with the assisted breathing device, such as connecting to the tubing, mask, endotracheal tube or other physical component of the assisted breathing device.

The embodiment in FIG. 43 can operate to create a pause at any point in the ventilation process, including an end inspiratory pause or an end expiratory pause. To create an end inspiratory pause, the controller will cause the device to follow the following steps:

1. During normal ventilator operation, valve 4302 is open, and valve 4308 is closed.

2. To create a pause at the end of inspiration, valve 4302 is closed after patient inspires.

3. The system pauses in this configuration for a period to collect COS data.

4. At the end of a preset time period, or when adequate COS data has been collected, valve 4308 is opened.

5. Valve 4308 remains open long enough for the patient to exhale his/her breath. This time frame may be preset, or may be determined based on the pressure or flow signal from sensor 4310, for example, when air flow or air pressure have reached a near zero level.

6. After the patient has exhaled most or all of his/her breath, valve 4308 is again closed and valve 4302 is opened, and normal ventilator function is resumed.

This embodiment allows the airway device to create a pause in the ventilator function delivered to the patient without affecting the intrinsic function of the ventilator itself, or without affecting the function of the ventilator to the extent of causing an error or interrupting the ventilator function for the patient. An end expiratory pause may be created similarly as follows:

1. During normal ventilator operation, valve 4302 is open, and valve 4308 is closed.

2. To create a pause at the end of expiration, valve 4302 is closed after patient exhales.

3. The system pauses in this configuration for a period to collect COS data.

4. At the end of a preset time period, or when adequate COS data has been collected, valve 4308 is opened.

5. Valve 4308 remains open long enough for the patient to inhale his/her breath. This time frame may be preset, or may be determined based on the pressure or flow signal from sensor 4310, for example, when air flow or air pressure have reached a near zero level.

6. After the patient has inhaled most or all of his/her breath, valve 4308 is again closed and valve 4302 is opened, and normal ventilator function is resumed.

Note that the flow/pressure sensor may sense when to start step 2, so it may determine when the patient is at the end of an inhalation or an expiration. Alternatively, pressure sensor 3804 may be used for this function and any other functions of pressure/flow sensor 4310.

FIG. 44 shows an embodiment of the airway pressure device which performs similarly to the device in FIG. 43 with an additional valve, patient valve 4304. The controller of this embodiment performs the following steps to create a pause to collect COS data:

1. During normal ventilator operation, valve 4302 and 4304 are open, and valve 4308 is closed.

2. To create a pause at the end of inspiration, valve 4304 is closed after patient inspires.

3. The system pauses in this configuration for a period to collect COS data.

4. At the end of a preset time period, or when adequate COS data has been collected, valves 4304 and 4308 are opened and valve 4302 is closed.

5. Valve 4308 remains open long enough for the patient to exhale his/her breath. This time frame may be preset, or may be determined based on the pressure or flow signal from sensor 4310, for example, when air flow or air pressure have reached a near zero level.

6. After the patient has exhaled most or all of his/her breath, valve 4308 is again closed and valve 4302 is opened, and normal ventilator function is resumed.

The embodiment in FIG. 44 allows more control over the operation of the airway device and may also be operated to create an end expiration pause, similar to the device in FIG. 43. FIGS. 43 and 44 show embodiments which are in-line with the ventilation tube, but add-on versions, analogous to the embodiment shown in FIG. 39 are also envisioned.

In some embodiments, data collected from the airway device may be used to control other medical devices/equipment. For example, airway device data may be analyzed and the results used to control drug delivery, fluid infusion, assisted ventilation parameters, CPAP parameters, etc.

In some embodiments, the airway device may be incorporated into a tracheotomy tube, a chest tube, or other device which accesses the lungs or has pleural access. Some devices which incorporate the airway device may access the lungs via the nose, or through the chest.

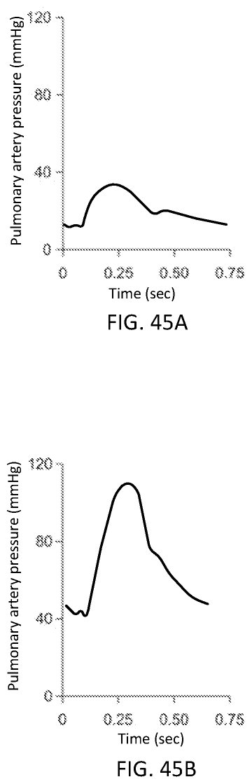

In some embodiments, the airway device may be used to help diagnose CTEPH (Chronic Thromboembolic Pulmonary Hypertension), or other forms of pulmonary hypertension. The COS signal in pulmonary hypertension patients will be different than the COS signal of patients without pulmonary hypertension. For example, the COS signal is likely to be higher (a higher pressure value) and the magnitude of the pressure pulse is likely to be greater. FIG. 45A represents the pulmonary artery pressure curve of a healthy patient, where FIG. 45B represents the pulmonary artery pressure curve of a patient with pulmonary hypertension. The COS curve is related to the pulmonary artery pressure curve and will show a similar difference in pulmonary hypertension patients. This difference in the COS curve may help identify patients with pulmonary hypertension, and specifically, CTEPH, so that they can get treatment.

Some embodiments of the airway device are specifically configured to predict, diagnose, and monitor lung conditions such as pneumonia. Many of the sensors disclosed herein collect data relating to pneumonia. For example, at least the following sensed data may be used to predict, diagnose and manage pneumonia:

| Sensor | Parameter received |
|---|---|
| Pulse oximeter/photoplethysmograph | Blood oxygen level |
| | Heart rate |
| Microphone | Lung sounds/breathing sounds |
| | Respiratory rate |
| Temperature sensor | Body temperature |
| Spirometer | Pulmonary function, including: |
| | Inhalation/exhalation volume |
| | Inhalation/exhalation flow |
| | Forced expiratory volume |
| | Forced vital capacity |
| | Lung capacity |
| | Respiratory rate |

The sensors above (as well as others) may be used to collect the parameters above which may be used to:
predict pneumonia
diagnose pneumonia
manage pneumonia (assess its progress)

The controller of the device may communicate information relating to the prediction, diagnoses, and/or management/progress of pneumonia to the user, to the user's doctor, or elsewhere. The communication may come in the form of an alert, including visual, audible and/or tactile, or the communication may come in the form of a text or email or other communication. The alert may be delivered to the device itself, to a user's mobile phone/computer/tablet and/or remotely, to a remote server, remote doctor, remote health or medical record, etc.

For example, the blood oxygen level may decrease (a deviation from normal or baseline), or surpass a threshold level as pneumonia develops. For example, a pulse oximetry level of less than 94% may indicate that pneumonia has developed. For example, a pulse oximetry level of less than 96% may indicate that pneumonia has developed. For example, a pulse oximetry level of less than 98% may indicate that pneumonia has developed. A decrease or increase decrease in oxygen level over time may indicate pneumonia developing or improving, respectively. Normal blood oxygen level may be around 95-96%%, but a person's normal may be different and therefor a deviation from the person's baseline may be considered abnormal.

Heart rate may also be factored into the pneumonia assessment. A greater heart rate (or a deviation from normal or baseline), or a heart rate that surpasses a threshold level may indicate a greater risk for developing pneumonia, may indicate that pneumonia has developed, or may be used to manage pneumonia. For example, a heart rate of greater than about 100 beats/minute may be an indicator that pneumonia has developed. For example, a heart rate of greater than about 105 beats/minute may be an indicator that pneumonia has developed. For example, a heart rate of greater than about 108 beats/minute may be an indicator that pneumonia has developed. For example, a heart rate of greater than about 110 beats/minute may be an indicator that pneumonia has developed. An increased heart rate over time may indicate that pneumonia is developing, or a decrease in heart rate over time may indicate that the user is recovering from pneumonia. Normal heart rate may be less than around 100 beats per minute, but a person's normal may be different and therefor a deviation from the person's baseline may be considered abnormal.

Abnormal lung sounds, such as crackling or bubbling noises/rale, may increase, or surpass a threshold level as pneumonia develops. Other sounds that may be detected which may be related to the development of pneumonia are thuds heard when the chest is tapped, pleural friction rub sounds, lack of breath sounds, wheezing, egophony, asymmetric sounds etc. An increase or decrease in abnormal lung sounds over time may indicate pneumonia developing or improving, respectively.

Respiratory rate may also be used to predict, diagnose and/or manage pneumonia. For example, a deviation from normal or baseline, or a respiratory rate surpassing a threshold level, may be considered in predicting, diagnosing and/or managing pneumonia. For example, a respiratory rate above around 25 breaths/minute may indicate that pneumonia has developed. An increase or decrease in respiratory rate over time may indicate pneumonia developing or improving, respectively. Normal respiratory rate may be less than around 20-25 breaths per minute, and greater than around 12 beats per minute, but a person's normal may be different and therefor a deviation from the person's baseline may be considered abnormal.

The user's temperature may increase (or deviate from normal or baseline), or surpass a threshold level, as pneumonia develops. Measured temperature above a certain threshold may be an indication that pneumonia has developed. Measured temperature above at the same, or a different, threshold for a certain period of time may be an indication that pneumonia has developed. For example, a temperature threshold of around 100 degrees Fahrenheit may indicate pneumonia. For example, a temperature threshold of around 100.4 degrees Fahrenheit may indicate pneumonia. For example, a temperature threshold of around 101 degrees Fahrenheit may indicate pneumonia. For example, a temperature threshold of around 102 degrees Fahrenheit may indicate pneumonia. For example, a temperature threshold of around 103 degrees Fahrenheit may indicate pneumonia. For example, a temperature threshold of around 104 degrees Fahrenheit may indicate pneumonia. For example, a temperature threshold of around 105 degrees Fahrenheit may indicate pneumonia. Normal temperature may be below around 98.6 degrees Fahrenheit, but a person's normal may be different and therefor a deviation from the person's baseline may be considered abnormal.

The airway device may keep track of how long the user's temperature is above a certain threshold. For example, if the user's temperature is elevated above normal for 3 days or more, this may be indication that pneumonia has developed. Or, if the user's temperature is elevated above any of the thresholds disclosed above for 3 days or more, or 2 days or more, this may indicate that pneumonia has developed.

Temperatures which are increasing, but below the threshold, may indicate pneumonia is developing. The user's end expiratory temperature may be used, or temperature may be measured elsewhere, such as in side the mouth, on the forehead, hands, etc. An increase or decrease in temperature over time may indicate pneumonia developing or betting better.

Combinations of these parameters, and/or changes in these parameters may be used to predict, diagnose and/or manage pneumonia.

For example, a combination of temperature, fever duration, abnormal lung sounds and decreased blood oxygen levels may indicate the development of pneumonia. For example, the following combination may be indicative of pneumonia: a peak temperature within 24 hours of 40 degrees Celsius or more, a fever duration of 3 days or more, abnormal breath sounds, and a SpO2 of 96% or less. Other factors may also be considered in this combination.

For example, a combination of temperature, heart rate and pulse oximetry may indicate the development of pneumonia. For example, the following combination may be indicative of pneumonia: a temperature over 100.4 degrees Fahrenheit, a heart rate of greater than 110 beats/minute, and a pulse oximetry of less than 96%. Other factors may also be considered in this combination. For example, the following combination may be indicative of pneumonia: a temperature over 99.9 degrees Fahrenheit, a heart rate of greater than 108 beats/minute, and a pulse oximetry of less than 94%. Other factors may also be considered in this combination.

For example, a combination of temperature, pulse rate, and lung sounds may indicate the development of pneumonia. For example, the following combination may be indicative of pneumonia: a temperature greater than 37.8 degrees Celsius, a heart rate of greater than 100 beats/min, and abnormal lung sounds. Other factors may also be considered in this combination, for example, the absence of asthma.

Thresholds and/or deviations from normal of the following parameters may also be used in the predicting, diagnosing and/or managing of pneumonia: inhalation volume, exhalation volume, inhalation flow, exhalation flow, forced expiratory volume, forced vital capacity, and/or lung capacity.

Thresholds for various physiologic parameters used in predicting, diagnosing and/or managing pneumonia may be absolute thresholds, or may be relative thresholds based on the user's baseline parameter measurements. For example, the temperature threshold may be 100.4 degrees Fahrenheit, or it may be 1.8 degrees Fahrenheit above the user's normal temperature, or baseline temperature.

Pneumonia may be predicted 3-5 days before pneumonia develops. Alternatively, pneumonia may be predicted 4-7 days before pneumonia develops. Alternatively, pneumonia may be predicted 1-2 days before pneumonia develops. Alternatively, pneumonia may be predicted 1-3 days before pneumonia develops. Alternatively, pneumonia may be predicted 1-4 days before pneumonia develops. Alternatively, pneumonia may be predicted 1-5 days before pneumonia develops. Alternatively, pneumonia may be predicted 1-5 days before pneumonia develops. Alternatively, pneumonia may be predicted 1-7 days before pneumonia develops.

The airway device may include an interface for a user to input data, via touchscreen or other user interface, or to receive data from a health record, computer, phone etc. For example, the airway device may receive information relating to the user's age, weight, symptoms (sore throat, pain, nausea, cough, asthma, dyspnea, chills, chest pain, vomiting, sweating, Rhinorrhea, myalgia, sputum, etc.), medical history (history of pneumonia, chronic cough, smoker, sputum production, chills, chest pain, sweats, alcoholism, drug use, bloody sputum, fever, etc.), etc.

The airway device may also include incentive spirometer functions to help with the recovery from pneumonia. For example, users may be prompted to breathe slow, deep breaths. Sensors such as pressure, flow, temperature sensors etc., and/or any of the other sensors mentioned herein, may be used to assess the user's breathing and prompt the user to breathe differently. The airway device may include a floating ball or disc to help the user control and maintain his/her breathing. Alternatively, the sensors may provide the user visual feedback, such as lights, sounds, vibration, to indicate whether the breathing is on target or not.

An attractive aspect of the airway device is the ability to predict, detect and/or manage pneumonia remotely, from home or elsewhere, and not necessarily in a hospital or medical facility, where contagion may be a risk. In some embodiments, the user may not need to enter a medical facility at all. For example, the data from the airway device may be shared with a physician, or other medical worker, such as a pharmacist, and when the risk of pneumonia crosses a certain threshold, the user may be prescribed antibiotics, or other medications. The sharing of data may be automatic, or by request of the user, or by request of a medical professional.

If pneumonia is caught early enough, and the user is prescribed antibiotics early enough, full fledge pneumonia may be avoided, or its severity lessened. In addition, if treatment by antibiotics can take place at home, there is less risk of contagion. The airway device may be able to determine if pneumonia has developed to the point where it is advisable that the user come into a hospital or other medical facility. By screening patients, the airway device can help keep the less sick patients home, treat them, and only admit the patients into a hospital who need hospitalization.

The airway device may also allow patients to leave a medical facility sooner, and with fewer complications and/or relapses, by monitoring the user's lung health after he/she has been released from the hospital. This monitoring may be similar to the assessment of pneumonia risk, but in reverse. For example, the airway device may be assessing patient recovery by sensing a decrease in body temperature, a decrease in lung sounds, an increase in blood oxygen level, a decrease in respiratory rate, a decrease in heart rate and an increase in lung functions. Similar to pneumonia prediction, and identification, management and/or recovery may be assessed by a combination of these and other parameters, including changes in these or a combination of these parameters over time.

A display in communication with the airway device may show the user his or her risk level for pneumonia, level of pneumonia, days with pneumonia, recovery level, etc. Although the disclosure above refers to pneumonia, the airway device may predict, identify and manage other conditions as well, including flu, viruses, contagious diseases etc. The same, or different health parameters may be used. For example, the airway device may be used to diagnose, and assess the seriousness of viruses such as a coronavirus (for example COVID-19), or flu viruses, to determine who should be hospitalized and who should not, in addition to predicting, identifying and managing people with these various conditions.

Some embodiments of the airway device are designed to work with current capnography systems, or incorporate capnography functions. These embodiments are of particular use with a patient which is on a ventilator, or other assisted breathing device.

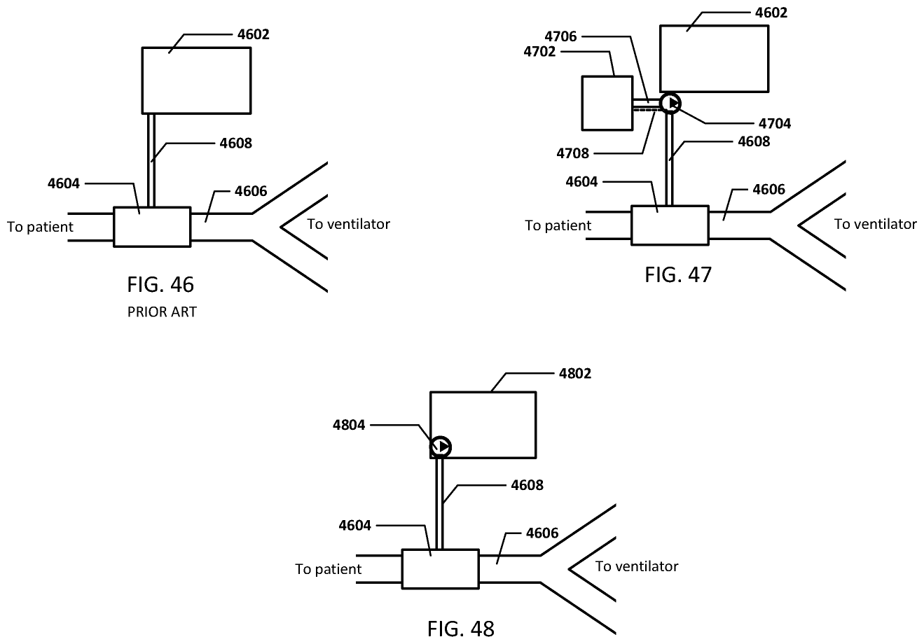

FIG. 46 shows a currently available capnography system. Shown here is capnography sensor and display 4602, capnography sampling adapter 4604 in line with ventilator tubing 4606 and capnography sampling tube 4608. The sampling adapter is in fluid communication with the ventilator tubing, as is the capnography sampling tube and the sensor within the capnography device. During breathing, respiratory gases flow through ventilator tubing 4606 and a portion of respiratory gas flows up capnography sampling tube 4608 and contacts a CO2 sensor in capnography device 4602. The capnography device senses CO2 in the ventilation tubing and displays the concentration or partial pressure of CO2 in the respiratory gases.

FIG. 47 shows the airway device incorporated with a capnography system. Shown here is airway device 4702, which is in fluid communication with capnography sampling tube 4608 via valve 4704 and airway device connection tube 4706. Valve 4704 is controlled via electrical connection between the valve and the airway device via connector 4708. In this embodiment, the airway device is spliced into the capnography system either at the junction between capnography sampling tube 4608 and capnography device 4602, between sampling adapter 4604 and capnography sampling tube 4608, or elsewhere. Valve 4704, controlled by the airway device, can be set to allow air flow to capnography device 4602, or airway device 4702, or, in some instances, both.

Generally, valve 4704 is set to allow air flow to either the capnography device or the airway device. This is to prevent contamination of the pressure or other data signals collected by the airway device. During use, valve 4704 may be generally open to capnography device 4602, and closed to airway device 4702. Periodically, the airway device may instruct valve 4704 to close fluid communication to capnography device 4602, and open fluid communication to airway device 4702. This orientation of the valve allows for the airway device to collect data related to the breathing of the patient. This collection may be for a short period of time, for example less than 10 seconds, less than 20 seconds, less than 30 seconds, less than 1 minutes, less than 3 minutes, less than 5 minutes etc. this collection may be on an automatic and periodic basis or on an on-demand basis. If on a periodic basis, the airway device may collect data at least every 5 minutes, every 10 minutes, every 30 minutes, every 60 minutes, or other time increment.

The airway data collection period may be a longer period of time, for example, 30 minutes, one hour, or greater than one hour.

In the embodiment shown in FIG. 47, the airway device may be used with any capnography system.

FIG. 48 shows a combination airway device/capnography system. In this embodiment, combination airway device and capnography system 4802 incorporate the functions of both the airway device and the capnography device disclosed in association with FIG. 47. Valve 4804 operates similarly to valve 4708 and may be incorporated into combination system 4802. In some embodiment of the combination airway device/capnography system, a valve may not be necessary.

In some embodiments, core body temperature is determined using exhalation temperature.

In some embodiments, an individual patient's baseline data is collected, and used to determine patient relative health in the future by comparing data to the baseline data.

Note that any features disclosed in association with any embodiment herein may be used with any other embodiment disclosed herein.

What is claimed is:

1. An apparatus for determining health of a user, comprising:
a housing configured to be held by the user when in use;
a mouthpiece in communication with the housing and configured for insertion within a mouth of the user when in use;
a temperature sensor positioned within or along the housing and configured for sensing a temperature of the user;
a pulse oximeter sensor positioned along the housing for contacting the user and sensing a heart rate or blood oxygen level of the user;
a microphone positioned within or along the housing for obtaining sounds associated with respiration from the user; and
a controller configured to receive physiologic parameters including the temperature, heart rate, blood oxygen level, and sounds associated with respiration and determine a likelihood of contracting pneumonia, a presence of pneumonia, or a status of pneumonia within the user based on deviations from a threshold value which are present in at least two of the physiologic parameters.

2. The apparatus of claim 1 further comprising an indicator in communication with the controller and configured to display or alert the user or a second user of the likelihood, presence, or status of pneumonia within the user.

3. The apparatus of claim 1 wherein a deviation in the temperature is present when the temperature is over 100.4 degrees F.

4. The apparatus of claim 1 wherein a deviation in the heart rate is present when the heart rate is over 110 beats per minute.

5. The apparatus of claim 1 wherein a deviation in the blood oxygen level is present when the blood oxygen level is less than 96%.

6. The apparatus of claim 1 wherein a deviation in the sounds associated with respiration are present when the sounds are abnormal.

7. The apparatus of claim 1 wherein the controller is configured to determine the likelihood of contracting pneumonia, the presence of pneumonia, or the status of pneumonia within the user when two of the following physiologic parameters are present: (i) the temperature over 100.4 degrees F.; (ii) the heart rate over 110 beats per minute; (iii) the blood oxygen level less than 96%; (iv) the sounds are abnormal.

8. The apparatus of claim 1 wherein the controller is configured to determine the likelihood of contracting pneumonia, the presence of pneumonia, or the status of pneumonia within the user when three of the following physiologic parameters are present: (i) the temperature over 100.4 degrees F.; (ii) the heart rate over 110 beats per minute; (iii) the blood oxygen level less than 96%; (iv) the sounds are abnormal.

9. The apparatus of claim 1 wherein the controller is configured to determine the likelihood of contracting pneumonia, the presence of pneumonia, or the status of pneumonia within the user when all of the following physiologic parameters are present: (i) the temperature over 100.4 degrees F.; (ii) the heart rate over 110 beats per minute; (iii) the blood oxygen level less than 96%; (iv) the sounds are abnormal.

10. The apparatus of claim 1 wherein the deviation in the temperature includes a presence of a fever for a period of time over the threshold value in the user.

11. The apparatus of claim 1 wherein the physiologic parameters further include a deviation in an inhalation volume of the user.

12. The apparatus of claim 1 wherein the physiologic parameters further include a deviation in an exhalation volume of the user.

13. The apparatus of claim 1 wherein the physiologic parameters further include a deviation in an inhalation flow of the user.

14. The apparatus of claim 1 wherein the physiologic parameters further include a deviation in an exhalation flow of the user.

15. The apparatus of claim 1 wherein the physiologic parameters further include a deviation in a forced expiratory volume of the user.

16. The apparatus of claim 1 wherein the physiologic parameters further include a deviation in a forced vital capacity of the user.

17. The apparatus of claim 1 wherein the physiologic parameters further include a deviation in a lung capacity of the user.

18. The apparatus of claim 1 wherein the physiologic parameters further include a deviation in a respiratory rate of the user.

19. The apparatus of claim 1 wherein the controller is further configured to account for an age of the user in determining the likelihood, presence, or status of pneumonia in the user.

20. The apparatus of claim 1 wherein the physiologic parameters further include a presence or absence of a sore throat in the user.

21. The apparatus of claim 1 wherein the controller is further configured to account for a history of pneumonia of the user in determining the likelihood, presence, or status of pneumonia in the user.

22. The apparatus of claim 1 further comprising a pressure sensor positioned within or along the housing for measuring cardiogenic oscillations from the user.

23. The apparatus of claim 22 wherein the controller is further configured to determine a cardiogenic oscillation waveform from the cardiogenic oscillations measured from the user and determine for presence of a deviation in the cardiogenic oscillation waveform.

24. The apparatus of claim 1 wherein the housing is configured as a handpiece.

25. The apparatus of claim 1 wherein the housing is configured to be portable by the user.

26. A method of determining health of a user, comprising:
sensing a temperature of a user via a temperature sensor positioned within or along a housing;
sensing a heart rate or blood oxygen level of the user via a pulse oximeter sensor positioned along the housing;
sensing sounds associated with respiration of the user via a microphone positioned within or along the housing;
receiving into a controller physiologic parameters including the temperature, heart rate, blood oxygen level, and sounds associated with respiration; and
determining via the controller a likelihood of contracting pneumonia, a presence of pneumonia, or a status of pneumonia within the user based on deviations from a threshold value which are present in at least two of the physiologic parameters.

27. The method of claim 26 wherein sensing sounds comprises inserting a mouthpiece in communication with the housing within a mouth of the user when in use.

28. The method of claim 26 further comprising displaying or alerting the user or a second user of the likelihood, presence, or status of pneumonia within the user.

29. The method of claim 26 wherein determining via the controller comprises determining whether a deviation in the temperature is present when the temperature is over 100.4 degrees F.

30. The method of claim 26 wherein determining via the controller comprises determining whether a deviation in the heart rate is present when the heart rate is over 110 beats per minute.

31. The method of claim 26 wherein determining via the controller comprises determining whether a deviation in the blood oxygen level is present when the blood oxygen level is less than 96%.

32. The method of claim 26 wherein determining via the controller comprises determining whether a deviation in the sounds associated with respiration are present when the sounds are abnormal.

33. The method of claim 26 wherein determining via the controller comprises determining the likelihood of contracting pneumonia, the presence of pneumonia, or the status of pneumonia within the user when two of the following physiologic parameters are present: (i) the temperature over 100.4 degrees F.; (ii) the heart rate over 110 beats per minute; (iii) the blood oxygen level less than 96%; (iv) the sounds are abnormal.

34. The method of claim 26 wherein determining via the controller comprises determining the likelihood of contracting pneumonia, the presence of pneumonia, or the status of pneumonia within the user when three of the following physiologic parameters are present: (i) the temperature over 100.4 degrees F.; (ii) the heart rate over 110 beats per minute; (iii) the blood oxygen level less than 96%; (iv) the sounds are abnormal.

35. The method of claim 26 wherein determining via the controller comprises determining the likelihood of contracting pneumonia, the presence of pneumonia, or the status of pneumonia within the user when all of the following physiologic parameters are present: (i) the temperature over 100.4 degrees F.; (ii) the heart rate over 110 beats per minute; (iii) the blood oxygen level less than 96%; (iv) the sounds are abnormal.

36. The method of claim 26 wherein the deviation in the temperature includes a presence of a fever for a period of time over the threshold value in the user.

37. The method of claim 26 wherein receiving into the controller physiologic parameters further comprises receiving data relating to an inhalation volume of the user.

38. The method of claim 26 wherein receiving into the controller physiologic parameters further comprises receiving data relating to an exhalation volume of the user.

39. The method of claim 26 wherein receiving into the controller physiologic parameters further comprises receiving data relating to an inhalation flow of the user.

40. The method of claim 26 wherein receiving into the controller physiologic parameters further comprises receiving data relating to an exhalation flow of the user.

41. The method of claim 26 wherein receiving into the controller physiologic parameters further comprises receiving data relating to a forced expiratory volume of the user.

42. The method of claim 26 wherein receiving into the controller physiologic parameters further comprises receiving data relating to a forced vital capacity of the user.

43. The method of claim 26 wherein receiving into the controller physiologic parameters further comprises receiving data relating to a lung capacity of the user.

44. The method of claim 26 wherein receiving into the controller physiologic parameters further comprises receiving data relating to a respiratory rate of the user.

45. The method of claim 26 wherein determining via the controller further comprises accounting for an age of the user in determining the likelihood, presence, or status of pneumonia in the user.

46. The method of claim 26 wherein receiving into the controller physiologic parameters further comprises receiving data relating to a presence or absence of a sore throat in the user.

47. The method of claim 26 wherein determining via the controller further comprises accounting for a history of pneumonia of the user in determining the likelihood, presence, or status of pneumonia in the user.

48. The method of claim 26 further comprising sensing a pressure of cardiogenic oscillations from the user via a pressure sensor positioned within or along the housing.

49. The method of claim 26 further comprising determining a cardiogenic oscillation waveform from the cardiogenic oscillations measured from the user and further determining for presence of a deviation in the cardiogenic oscillation waveform.

50. The method of claim 26 wherein the housing is configured as a handpiece.
